(19) United States
(12) Patent Application Publication
(10) Pub. No.: US 2011/0195995 A1
(43) Pub. Date: Aug. 11, 2011

(54) Methods of Optimizing Treatment of Estrogen-Receptor Positive Breast Cancers

(76) Inventors: James L. Wittliff, Louisville, KY (US); Daniel Allan Kerr, II, Louisville, KY (US)

(21) Appl. No.: 13/123,158

(22) PCT Filed: Oct. 13, 2009
(86) PCT No.: PCT/US2009/060506

§ 371 Date: Apr. 7, 2011

Related U.S. Application Data
(60) Provisional application No. 61/105,297, filed on Oct. 14, 2008.

(51) Int. Cl. A61K 31/4535; C12Q 1/68; A61K 31/135; A61P 35/00

(52) U.S. Cl. 514/326; 435/6.14; 514/646

(57) ABSTRACT

Estrogen-receptor positive and progesterone-receptor positive breast cancer treatment can be optimized by determining the level of expression of genes in a breast sample from a human having an estrogen-receptor positive and progesterone-receptor positive breast cancer that identify a human with an increased likelihood of recurrence of the breast cancer.

### RELATED APPLICATION

This application claims the benefit of U.S. Provisional Application No. 61/105,297, filed on Oct. 14, 2009, the entire teachings of which are incorporated herein by reference.

### BACKGROUND OF THE INVENTION

Breast cancer is a major health concern and one of the most prevalent forms of cancer in woman. Determining whether breast cancer has an increased likelihood of recurrence in a patient can effect the type and duration of treatment that may be administered to the patient, such as treatment with hormone therapy alone or in combination with chemotherapy and radiation therapy, which can, in turn, decrease mortality. Currently, techniques to assess an increased likelihood of recurrence of breast cancer in a patient, include the determination of gene expression patterns. However, such methods may be inaccurate, require the need to assess the expression of relatively large numbers of genes and may be inadequate to guide therapy options. Thus, there is a need to develop new, improved and effective methods of identifying a woman having an increased likelihood of recurrence of breast cancer, which may determine a course of therapy selection and prognosis.

### SUMMARY OF THE INVENTION

The present invention related to methods of optimizing treatment of a human having an estrogen-receptor positive breast cancer.

In an embodiment, the invention is a method of optimizing treatment of a human having an estrogen-receptor positive breast cancer, comprising the step of determining a level of expression of genes selected from the group consisting of ESR1, BCL-2 α, ERBB4, ERBB4 JM-a, RERG, CD34, EDG-1, NQO-1, PGR and PTGDS in a breast cancer tissue sample from the human, wherein underexpression of PGR, ERBB4 JM-a, RERG, CD34, EDG-1 and NQO-1 in the sample in combination with overexpression of ESR1, BCL-2 α, ERBB4 and PTGDS in the sample thereby identifies a human that would potentially benefit from a therapy that is alternative to or in combination with a selective estrogen receptor modulator (SERM).

In another embodiment, the invention is a method of optimizing treatment of a human having an estrogen-receptor positive breast cancer, comprising the step of determining a level of expression of genes selected from the group consisting of BCL-2 α, CA XII, ERBB4 and RERG in a breast cancer tissue sample from the human, wherein underexpression of the genes in the sample thereby identifies a human that has an increased likelihood of recurrence of the estrogen-receptor breast cancer that would potentially benefit from a therapy to decrease the likelihood of recurrence of the estrogen-receptor positive breast cancer.

In a further embodiment, the invention is a method of optimizing treatment of a human having an estrogen-receptor positive and progesterone-positive breast cancer, comprising the step of determining a level of expression of genes selected from the group consisting of PGR, CA XII, ERBB4, ERBB4 JM-a and RERG in a breast cancer tissue sample of the human, wherein underexpression of the genes in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

In yet another embodiment, the invention is a method of optimizing treatment of a human having an estrogen-receptor positive and progesterone-receptor positive breast cancer, comprising the step of determining a level of expression of genes selected from the group consisting of HER2, CA XII, ERBB4 JM-a and LIV 1 in a breast cancer tissue sample of the human, wherein the breast tissue sample is at least one member selected from the group consisting of a stage 1 breast cancer tissue sample and a stage 2 breast cancer tissue sample; and underexpression of CA XII and ERBB4 JM-a in the sample in combination with overexpression of HER2 and LIV1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and progesterone-receptor positive breast cancer.

In an additional embodiment, the invention is a method of optimizing treatment of a human having an estrogen-receptor positive and progesterone-receptor positive breast cancer, comprising the step of determining a level of expression of genes selected from the group consisting of ESR1, CA XII, ERBB4, CD34 and EDG1 in a breast cancer tissue sample of the human that is node-negative for the estrogen-receptor positive and progesterone-receptor positive breast cancer, wherein overexpression of ESR1 and CD34 in the sample in combination with underexpression of CA XII, ERBB4 and EDG1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

In still another embodiment, the invention is a method of optimizing treatment of a human having an estrogen-receptor positive and progesterone-receptor positive breast cancer, comprising the step of determining a level of expression of genes selected from the group consisting of CA XII, ERBB4, LIV1, CD34, EDG1 and NQO1 (optionally also ESR1) in a breast cancer tissue sample of the human that is node-negative for the estrogen-receptor positive and progesterone receptor positive breast cancer, wherein overexpression of LIV1, CD34 and NQO 1 in the sample in combination with underexpression of CA XII, ERBB4 and EDG1 in the sample thereby identifies a human that would potentially benefit from a therapy to increase the likelihood of survival of the human.

Another embodiment of the invention is a method of optimizing treatment of a human having an estrogen-receptor positive and progesterone-receptor positive breast cancer, comprising the step of determining a level of expression of genes selected from the group consisting of HER2, BCL2a, CA XII, CD34, EDG1 and NQO1 in a breast cancer tissue sample of the human that is lymph node-positive for the estrogen-receptor positive and progesterone-receptor positive breast cancer, wherein overexpression of HER2, BCL2a and EDG1 in the sample in combination with underexpression of CD34, CA XII and NQO1 in the sample thereby identifies a human that has an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

In yet another embodiment, the invention is a method of optimizing treatment of a human having an estrogen-receptor positive and progesterone-receptor positive breast cancer, comprising the step of determining a level of expression of genes selected from the group consisting of ERBB4 JM-a, CD34 and EDG1 in a breast cancer tissue sample of the human that is lymph node-positive for the estrogen-receptor positive and progesterone-receptor positive breast cancer, wherein overexpression of EDG1 in the sample in combination with underexpression of ERBB4 JM-a and CD34 in the sample thereby identifies a human that would potentially benefit from a therapy to increase the likelihood of survival of the human.

The methods of the invention can be employed to optimize treatment of an estrogen-receptor positive breast cancer in a human. Advantages of the claimed invention include, for example, relatively rapid determination of changes in gene expression on small amounts of tissue (e.g., fresh or frozen biopsies) by detecting changes in relatively few genes (e.g., 9, 6, 5, 4), which can improve the accuracy of identifying humans with an increased risk of recurrence of the breast cancer. The claimed methods can be employed in optimizing treatment of breast cancer thereby avoiding recurrence of the disease, serious illness consequent the disease and death.

### BRIEF DESCRIPTION OF THE FIGURES

FIGS. 1A and 1B depict Kaplan-Meier analyses of disease-free (FIG. 1A) and overall (FIG. 1B) survival probabilities of 4 breast cancer molecular subtypes determined by microarray analyses of gene expression using LCM-procured cancer cells. Two distinct subtypes were associated with estrogen-receptor positive (ER+) cancers (subtypes A and B) and two subtypes were associated with estrogen-receptor negative (ER−) cancers (subtypes C and D).

FIGS. 2A and 2B depict Kaplan-Meier regression analyses illustrating distinct survival probabilities for breast cancer molecular subtypes. Disease Free Survival (DFS) and Overall Survival (OS).

FIGS. 3A and 3B depict distribution of estrogen-receptor (ER) and progesterone-receptor (PR) protein levels within breast cancer subtypes. Mean ER protein levels are 359 fmol/mg protein for subtype A and 195 fmol/mg protein for subtype B. Mean progesterone receptor (PR) protein levels are 416 fmol/mg protein for subtype A and 228 fmol/mg protein for subtype B.

FIGS. 4A, 4B, 4C and 4D depict representative regression analyses illustrating relative gene expression obtained independently by qPCR and by microarray in 92 specimens.

FIGS. 5A and 5B depict Kaplan-Meier regression analyses of disease-free (FIG. 5A) and overall (FIG. 5B) survival probabilities in 210 ER+, stage 1-2 primary breast cancers according to PR gene expression.

FIGS. 6A and 6B depict Kaplan-Meier regression analyses of disease-free (FIG. 6A) and overall (FIG. 6B) survival probabilities in 180 ER+/PR+, stage 1-2 primary breast cancers according to multivariate Cox models.

FIGS. 7A and 7B depict Kaplan-Meier regression analyses of disease-free (FIG. 7A) and overall (FIG. 7B) survival probabilities in 111 ER+/PR+, node-negative primary breast cancers according to multivariate Cox models.

FIGS. 8A and 8B depict Kaplan-Meier regression analyses of disease-free (FIG. 8A) and overall (FIG. 8B) survival probabilities in 69 ER+/PR+, node-positive primary breast cancers according to multivariate Cox models.

FIGS. 9A and 9B depict Kaplan-Meier regression analyses of disease-free (FIG. 9A) and overall (FIG. 9B) survival probabilities in the ER+/PR+ population (n=180) as a function of nodal status and the multivariate gene models.

FIGS. 10A and 10B depict Kaplan-Meier regression analyses of DFS (FIG. 10A) and OS (FIG. 10B) in 60 ER+/PR+, stage 1-2 patients who received adjuvant Tamoxifen™ therapy. Risk groups were defined by applying multivariate Cox regression to the gene expression data using DFS as the endpoint.

FIG. 11 depicts estrogen-receptor positive and progesterone-receptor positive breast cancers for which treatment can be optimized by the methods of the invention.

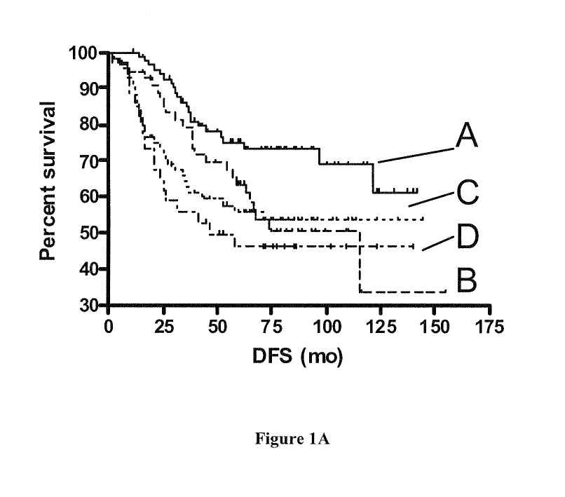

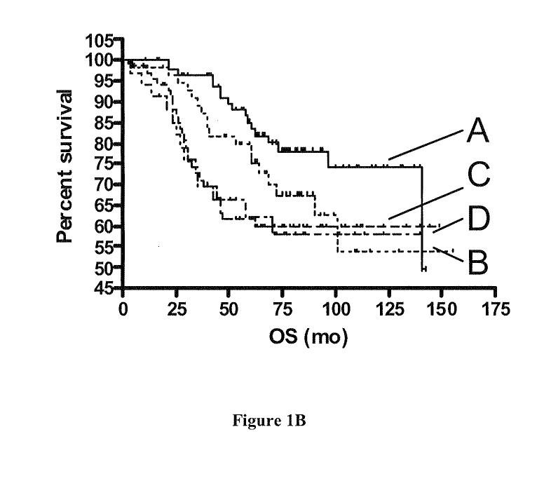

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The methods described herein are generally directed to methods of optimizing treatment of a human with an estrogen-receptor (ER, also referred to herein as “ESR” when referencing gene expression of the estrogen receptor) positive (ER+) breast cancer and ER+ and progesterone-receptor (PR, also referred to herein as “PGR” when referencing gene expression of the progesterone receptor) positive (PR+) breast cancers. Generally, breast cancers that are positive for both ER and PR have a decreased incidence of recurrence and, in some instances, decreased mortality. The methods described herein can facilitate critical and careful clinical management of optimal treatment of humans with breast cancers that are positive for both ER and PR, which decreases the likelihood of recurrence of the breast cancer and death consequent to the breast cancer.

In an embodiment, the invention is a method of optimizing treatment of a human having an estrogen-receptor positive breast cancer, comprising the step of determining a level of expression of genes selected from the group consisting of ESR1, BCL-2 α, ERBB4, ERBB4 JM-a, RERG, CD34, EDG-1 (also referred to herein as “EDG1”), NQO-1, PGR and PTGDS in a breast cancer tissue sample from the human, wherein underexpression of PGR, ERBB4 JM-a, RERG, CD34, EDG-1 and NQO-1 in the sample in combination with overexpression of ESR1, BCL-2 α, ERBB4 and PTGDS in the sample thereby identifies a human that would potentially benefit from a therapy that is alternative to or in combination with a selective estrogen receptor modulator, such as a 2-[4-[1,2-di(phenyl)but-1-enyl]phenoxy]-N,N-dimethylethanamine therapy (e.g., Tamoxifen™ therapy).

The ESR measured can be expression of at least one member selected from the group consisting of ESR1 (also referred to as “estrogen receptor alpha”) gene expression and ESR2 (also referred to as “estrogen receptor beta”) gene expression.

“Optimizing treatment,” as used herein, means identifying a therapy (e.g., chemotherapy, radiation therapy or any combination of therapies) that has the greatest chance of eliminating the breast cancer or causing remission of the breast cancer as detected by, for example, the presence of breast cancer cells in biopsies, and preventing metastasis of the breast cancer. Malignant breast tumors can form metastases to non-breast tissues and organs by entering the systemic circulatory system (arteries, veins) or lymphatic circulatory system. The methods described herein can be employed to optimize treatment to prevent or minimize metastases of a malignant breast tumor.

“Estrogen-receptor positive breast cancer,” as used herein, means that the levels of estrogen receptor protein in the breast cancer sample or biopsy are greater than about 10 fmol/mg protein (e.g., about 10 fmol/mg protein by ligand binding assay or about 15 fmol/mg protein by EIA) by established techniques, such as at least one member selected from the group consisting of radioligand binding, Enzyme ImmunoAssay (EIA) and semi-quantitative immunohistochemical assay (see, for example, Wittliff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W. B. Saunders Co. (1998)).

In an embodiment, the methods of the invention determine (measure) expression of genes in breast cancer samples from a human who has estrogen-dependent breast cancer. In another embodiment, the methods of the invention determine (measure) expression of genes in breast cancer sample from a human that has an estrogen-receptor positive breast cancer that is also progesterone-receptor positive breast cancer, which is also referred to herein as an “estrogen-receptor positive and a progesterone-receptor positive” or “ER⁺/PR⁺” breast cancer.

“Progestin-receptor positive breast cancer,” as used herein, means that the levels of progestin receptor protein in the breast cancer sample or biopsy measure greater than about 10 fmol/mg protein (e.g., about 10 fmol/mg protein by ligand binding assay or about 15 fmol/ng by EIA) by established techniques, such as at least one member selected from the group consisting of radioligand binding, EIA and semi-quantitative immunohistochemical assay (see, for example, Witttiff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W. B. Saunders Co. (1998)).

“Alternative therapy,” as used herein, means a treatment other than treatment with 2-[4-[1,2-di(phenyl)but-1-enyl]phenoxy]-N,N-dimethylethanamine therapy (i.e., Tamoxifen™ IUPAC designation (Z)-2-(para-(1,2-Dephenyl-1-butenyl)pehnoxyl)-N,N-dimethylamine) also referred to as Nolvadex™. “Alternative therapy,” is also referred to herein a “therapy that is alternative to.” The alternative therapy can be administered alone or in combination (e.g., before, during or after) with the estrogen-receptor antagonist 2-[4-[1,2-di(phenyl)but-1-enyl]phenoxy]-N,N-dimethylethanamine.

The methods described herein can further include the step of administering at least one alternative therapy to the human alone or in combination with the 2-[4-[1,2-di(phenyl)but-1-enyl]phenoxy]-N,N-dimethylethanamine therapy, thereby treating the human for the estrogen-receptor positive breast cancer. An exemplary alternative therapy can include at least one aromatase inhibitor (Mauri, D., et al., J. Natl. Cancer Inst. 98:1285-1291 (2006)) (e.g., Anastrozol, Arimidex™, 2-[3-(1-cyanol-1-methyl-ethyl)-5-(1H-1,2,4-triazol-1-ylmethyl)phenyl]-2-methyl-propanenitrile). Selective estrogen receptor modulator, for example, 2-(para-((Z)-4-chloro-1,2-diphenyl-1-butenyl)phenoxy)-N,N-dimethylethylamine, IUPAC designation) (Pagani, O., et al., Ann. Oncol. 15:1749-1759 (2004)) (Toremifene™) and [6-hydrox-2-(4-hydroxyphenyl)-1-benzothiophen-3-yl]-4-(2-piperidin-1-ium-1-ylethoxy)phenyl]methanone chloride (Raloxifene™, EVISTA® IUPAC designation (2-(4-Hydroxyphenyl)-6-hydroxybenzo(b)thien-3-yl)(4-(2-(1-piperidinyl)ethoxy)phenyl)methanone may be considered.

An alternative therapy can include administration of a compound that degrades the estrogen receptor (i.e., is not a estrogen receptor ligand), such as Fulvestrant™ (C₃₂H₄₇F₅O₃S or 13-methyl-7-[9-(4,4,5,5,5-pentafluoropentylsulfinyl)nonyl]-7,8,9,11,12,13,14,15,16,17-decahydro-6H-cyclopenta[a]phenanthrene-3,17-diol.

“Selective estrogen receptor modulator (SERM),” as used herein, refers to nonsteriodal and steriodal compounds that interact with the estrogen receptor to thereby affect or mediate the action of estrogens, such as 17β-estradiol. The administration of a SERM may provide the benefits of estrogens without the potentially adverse risk of increased cell proliferation in estrogen-responsive tissues, such as breast and uterine epithelium.

“Would potentially benefit,” as used herein, means that the breast cancer will go into remission, is substantially be eliminated or palliative remediation of the disease in the human.

In another embodiment, the invention is a method of optimizing treatment of a human having an estrogen-receptor positive breast cancer, comprising the step of determining a level of expression of genes selected from the group consisting of BCL-2 α, CAXII, ERBB4 and RERG in a breast cancer tissue sample from the human, wherein underexpression of the genes in the sample thereby identifies a human that has an increased likelihood of recurrence of the estrogen-receptor positive breast cancer that would potentially benefit from a therapy to decrease the likelihood of recurrence of the estrogen-receptor positive breast cancer.

The method of optimizing treatment of a human having an estrogen-receptor positive breast cancer, comprising the step of determining a level of expression of genes selected from the group consisting of BCL-2 α, CA XII, ERBB4 and RERG in a breast cancer tissue sample from the human, can further include the step of determining expression of PGR in the sample, wherein underexpression of PGR in the sample in combination with underexpression of BCL-2 α, CA XII, ERBB4 and RERG identifies a human that would benefit from the therapy. The human may have an increased likelihood of recurrence of the breast cancer. In an embodiment, the ERBB4 gene is an JM-a ERBB4 variant.

In a further embodiment, the invention is a method of optimizing treatment of a human having an estrogen-receptor positive breast cancer, comprising the step of determining a level of expression of genes selected from the group consisting of BCL-2 α, CA XII, ERBB4 and RERG in a breast cancer tissue sample from the human, wherein underexpression of the genes in the sample thereby identifies a human that has an increased likelihood of recurrence of the estrogen-receptor positive breast cancer that would potentially benefit from a therapy to decrease the likelihood of recurrence of the estrogen-receptor positive breast cancer, wherein treatment of the human increases the likelihood of survival of the human.

“An increased likelihood of recurrence of breast cancer,” as used herein, means that the human had at least one incident of a diagnosis of breast cancer and has an elevated probability of having the breast cancer return. For example, in a meta-analysis (from seven different studies) of more than about 3,500 patients who had received some type of post-surgical adjuvant therapy for breast cancer, risk of cancer recurrence was greatest during the first two years following surgery. After this period, the research showed a steady decrease in the risk of recurrence until year five when the risk of recurrence declined slowly and averaged about 4.3% per year (Saphner T, et al., J Clin Oncol. 14:2738-2746 (1996)). Some proportion of breast cancer recurrences seen in this study occurred more than about five years after surgery, between about six to about 12 years after surgery, even in patients who typically would be considered at low risk for recurrence because their cancer had not spread to the lymph nodes at the time of diagnosis (node-negative). This study shows that through at least about 12 years of follow-up, the risk of breast cancer recurrence remains appreciable and even some patients considered low risk have some risk of the cancer recurring.

“Increased likelihood of survival,” as used herein, means that the human that had at least one incident of a diagnosis of breast cancer has an elevated probability of living.

In another embodiment, the invention is a method of optimizing treatment of a human having an estrogen-receptor positive and progesterone-positive breast cancer, comprising the step of determining a level of expression of genes selected from the group consisting of PGR, CA XII, ERBB4, ERBB4 JM-a and RERG in a breast cancer tissue sample of the human, wherein underexpression of the genes in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

In still another embodiment, the invention is a method of optimizing treatment of a human having an estrogen-receptor positive and progesterone-receptor positive breast cancer, comprising the step of determining a level of expression of genes selected from the group consisting of HER2, CA XII, ERBB4 JM-a and LIV1 (also referred to herein as “LIV-1”) in a breast cancer tissue sample of the human, wherein the breast tissue sample is at least one member selected from the group consisting of a stage 1 breast cancer tissue sample and a stage 2 breast cancer tissue sample; and underexpression of CA XII and ERBB4 JM-a in the sample in combination with overexpression of HER2 and LIV1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

In a further embodiment, the invention is a method of optimizing treatment of a human having an estrogen-receptor positive and progesterone-receptor positive breast cancer, comprising the step of determining a level of expression of genes selected from the group consisting of ESR1, CA XII, ERBB4, CD34 and EDG1 in a breast cancer tissue sample of the human that is node-negative for the estrogen-receptor positive and progesterone-receptor positive breast cancer, wherein overexpression of ESR1 and CD34 in the sample in combination with underexpression of CA XII, ERBB4 and EDG1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

Another embodiment of the invention is a method of optimizing treatment of a human having an estrogen-receptor positive and progesterone-receptor positive breast cancer, comprising the step of determining a level of expression of genes selected from the group consisting of CA XII, ERBB4, LIV1, CD34, EDG1 and NQO1 in a breast cancer tissue sample of the human that is node-negative for the estrogen-receptor positive and progesterone receptor positive breast cancer, wherein overexpression of LIV1, CD34 and NQO 1 in the sample in combination with underexpression of CA XII, ERBB4 and EDG1 in the sample thereby identifies a human that would potentially benefit from a therapy to increase the likelihood of survival of the human.

In still another embodiment, the invention is a method of optimizing treatment of a human having an estrogen-receptor positive and progesterone-receptor positive breast cancer, comprising the step of determining a level of expression of genes selected from the group consisting of CD34, HER2, BCL2a, CA XII, EDG1 and NQO1 in a breast cancer tissue sample of the human that is lymph node-positive for the estrogen-receptor positive and progesterone-receptor positive breast cancer, wherein overexpression of HER2, BCL2a and EDG1 in the sample in combination with underexpression of CD34, CA XII and NQO1 in the sample thereby identifies a human that has an increased likelihood of recurrence of the estrogen-receptor positive and progesterone-receptor positive breast cancer that would potentially benefit from a therapy to decrease the likelihood of recurrence of the estrogen-receptor positive and positive-receptor positive breast cancer.

An additional embodiment of the invention is a method of optimizing treatment of a human having an estrogen-receptor positive and progesterone-receptor positive breast cancer, comprising the step of determining a level of expression of genes selected from the group consisting of ERBB4 JM-a, CD34 and EDG1 in a breast cancer tissue sample of the human that is lymph node-positive for the estrogen-receptor positive and progesterone-receptor positive breast cancer, wherein overexpression of EDG1 in the sample in combination with underexpression of ERBB4 JM-a and CD34 in the sample thereby identifies a human that would potentially benefit from a therapy to increase the likelihood of survival of the human.

The methods described herein can further include the step of treating the human with a therapy that decreases the likelihood of recurrence of the breast cancer. The therapy may increase the likelihood of survival of the human. The selection of therapy will depend on, for example, the stage of the breast cancer, the expression of particular genes, age of the human, overall health status and current treatment. For example, polychemotherapy with at least 4 cycles of one member selected from the group consisting of cyclophosphamide in combination with methotrexate and fluorouracil (CMF); doxorubicin in combination with fluorouracil and cyclophosphamide (FAC); and fluoruracil in combination with epirubicin and cyclophosphamide (see, for example, Early Breast Cancer Trialists' Collaborative Group (EBCTCG), Lancet 365(9472):1687-717 (2005) may be used as a therapy to optimize treatment of humans with ER⁺ and PR⁺ breast cancers. Chemotherapy may be combined with radiation therapy and/or endocrine therapy, such as treatment with at least one member selected from the group consisting of at least one estrogen receptor antagonist, at least one aromatase inhibitor and at least one selective estrogen receptor modulator (“SERM”). Alternatively, to optimize treatment of the breast cancer, chemoendocrine therapies may be employed in combination with endocrine adjuvant therapies, for example, in humans identified by the methods of the invention that have lymph node negative breast cancers.

Radiation therapy, has generally be employed as a treatment for relatively large breast cancer tumors and breast cancers from humans with at least four (4) positive lymph nodes. Humans identified by the methods described herein that can potentially benefit from a therapy to decrease the likelihood of recurrence of the breast cancer, in particular cancers that are from lymph node-negative humans (also referred to herein as “patients”) may have optimized therapies that include more aggressive therapy, such as radiation even if the clinical profile, for example, small tumor, low lymph node involvement, would not otherwise lead itself to radiation therapy.

The methods of the invention can identify humans with increased risks of recurrence of the breast cancer can result in treatments that are customized to the patient and may be more clinically aggressive than patients who do not have an increased likelihood of recurrence of the breast cancer. Thus, treatment of humans having an increased likelihood of recurrence of the breast cancer can be a more aggressive therapy.

Regarding treatment of ER+/PR+, node-negative breast cancers (patients considered having good prognosis), adjuvant therapy selection may be minimal, depending upon the Oncologist's discretion. For example, adding only Tamoxifen™ maintenance therapy after surgical removal of the tumor is a viable option; however, many oncologists may choose to add a course of adjuvant chemotherapy (e.g., CMF, FAC, FEC). Employing the methods described herein, a patient can be identified that has a “high risk” of recurrence (i.e., the breast cancer sample has an expression profile of a particular gene subsets as described herein), indicating that the patient should receive more aggressive therapies (terms used by oncologists to describe, for example, dose escalations). Thus, a patient with the node-negative cancer would be a candidate for therapy regimens selected for patients with node-positive cancer, which include multiple courses of polychemotherapy and/or external beam radiation therapy. Various polychemotherapy regimens are used at the discretion of the oncologist depending upon the collective characteristics of the lesion, the patient parameters and health status and other features and would be within the knowledge and medical expertise of one skilled in the art. The regimens could include TAC (docetaxel plus doxorubicin and cyclophosphamide).

Thus, the methods of the invention can be employed to identify patients who are less likely to have a recurrence of a breast cancer. For example, overexpression of BCL-2 α, CA XII, ERBB4 and RERG gene expression, and optionally overexpression of PGR, in a breast cancer tissue sample from the human identifies a human that has a decreased likelihood of recurrence of the estrogen-receptor breast cancer that would potentially benefit from a less aggressive therapy to treat the breast cancer.

In an embodiment, the invention is a method of optimizing treatment of a human having an estrogen-receptor positive and progesterone-positive breast cancer, comprising the step of determining a level of expression of genes selected from the group consisting of PGR, CA XII, ERBB4, ERBB4 JM-a and RERG in a breast cancer tissue sample of the human, wherein overexpression of the genes in the sample thereby identifies a human having a decreased likelihood of recurrence of the estrogen-receptor positive and progesterone-receptor positive breast cancer that would potentially benefit from a less aggressive therapy to treat the estrogen-receptor positive and positive-receptor positive breast cancer.

In another embodiment, the invention is a method of optimizing treatment of a human having an estrogen-receptor positive and progesterone-receptor positive breast cancer, comprising the step of determining a level of expression of genes selected from the group consisting of CD34, HER2, CA XII, ERBB4 JM-a and LIV1 in a breast cancer tissue sample of the human, wherein the breast tissue sample is at least one member selected from the group consisting of a stage 1 breast cancer tissue sample and a stage 2 breast cancer tissue sample; and overexpression of CD34, CA XII and ERBB4 JM-a in the sample in combination with underexpression of HER2 and LIV1 in the sample thereby identifies a human having a decreased likelihood of recurrence of the estrogen-receptor positive and progesterone-receptor positive breast cancer that would potentially benefit from a less aggressive therapy to treat the estrogen-receptor positive and positive-receptor positive breast cancer.

In another embodiment, the invention is a method of optimizing treatment of a human having an estrogen-receptor positive and progesterone-receptor positive breast cancer, comprising the step of determining a level of expression of genes selected from the group consisting of ESR1, CA XII, ERBB4, CD34 and EDG1 in a breast cancer tissue sample of the human that is node-negative for the estrogen-receptor positive and progesterone-receptor positive breast cancer, wherein underexpression of ESR1 and CD34 in the sample in combination with overexpression of CA XII, ERBB4 and EDG1 in the sample thereby identifies a human having a decreased likelihood of recurrence of the estrogen-receptor positive and progesterone-receptor positive breast cancer that would potentially benefit from a less aggressive therapy to treat the estrogen-receptor positive and positive-receptor positive breast cancer.

Another embodiment of the invention is a method of optimizing treatment of a human having an estrogen-receptor positive and progesterone-receptor positive breast cancer, comprising the step of determining a level of expression of genes selected from the group consisting of HER2, BCL2a, CA XII, CD34, EDG1 and NQO1 in a breast cancer tissue sample of the human that is lymph node-positive for the estrogen-receptor positive and progesterone-receptor positive breast cancer, wherein underexpression of HER2, BCL2a and EDG1 in the sample in combination with overexpression of CA XII and NQO1 in the sample thereby identifies a human that has a decreased likelihood of recurrence of the estrogen-receptor positive and progesterone-receptor positive breast cancer that would potentially benefit from a less aggressive therapy to treat the estrogen-receptor positive and progesterone-receptor positive breast cancer.

In addition, ER+/PR+, node-positive cancers allows classification of certain patients within this group as “low risk” of recurrence, from the expression profile of a particular gene subset as described herein. Thus, even though the patient is node-positive, they may benefit from a less aggressive treatment (e.g., adjuvant Tamoxifen™ alone without additional polychemotherapy or radiation therapy).

The 10-gene model ESR1, PGR, BCL2α, ERBB4, ERBB4 JM-a, RERG, CD34, EDG1, NQ01 and PTGDS applied to ER+/PR+ breast cancer from patients treated with adjuvant Tamoxifen™ (regardless of nodal status) also separates patients into low or high risk groups. If a patient presents with a “high risk” gene signature determined by the invention, this indicates a likelihood of a poor response to adjuvant Tamoxifen™ therapy, and that patient is a candidate for more aggressive therapy (e.g., additional polychemotherapy and/or radiation therapy). The invention identifies patients with primary breast cancer likely to recur after Tamoxifen™ therapy compared to those patients with primary breast cancer who exhibit a long disease-free interval after Tamoxifen™ treatment.

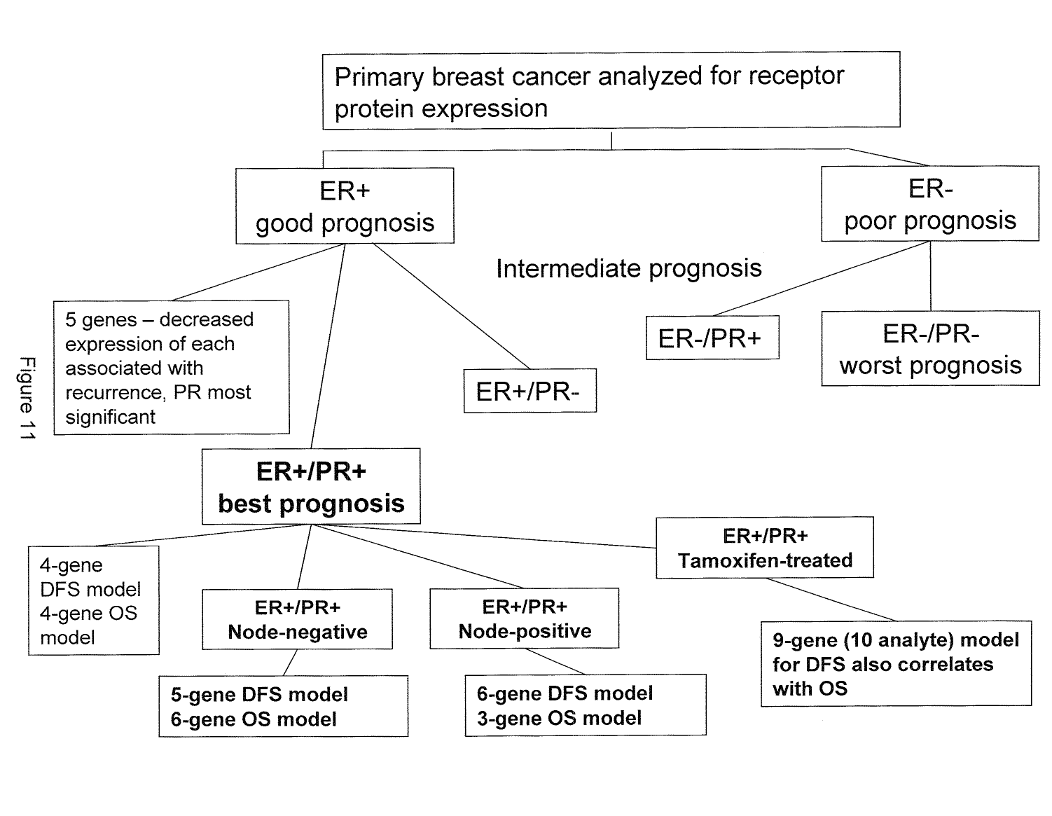

Thus, the expression of the genes described herein may predict the survival and prognosis of the human (see, for example, FIG. 11). For example, the methods described herein identify a human who has an increased likelihood of recurrence of breast cancer, which may indicate an increased likelihood of death. Likewise, employing the methods described herein, a human may be identified who has a relatively low likelihood of recurrence of breast cancer, which may indicate increased survival.

The methods of the invention can be employed to predict, for example, local recurrence of primary breast carcinoma and regional or distant metastases from primary breast carcinoma, which may provide prognostic evaluation of overall survival probabilities at time of diagnosis for primary breast carcinoma. The methods of the invention can be employed to optimize therapeutic regiments for treatment of the breast cancer, which would be customized to the patient by one of skill in the art based on factors such as age, health history, other disease and family history. The gene expression profiles described herein may provide biomarkers assessing disease progression and response in human cancers other than breast (e.g., ovarian, uterine, colon).

The methods described herein provide clinically relevant subset of genes in a tissue biopsy that predicts breast cancer behavior (gene subset of about 8 to about 15 genes is commercially feasible for development of a molecular diagnostic acceptable to clinicians, pathologists and laboratory medicine specialists. The methods of the invention may be performed quickly on tissue biopsies, and the entire panel of genomic biomarkers may be measured simultaneously in conventional formats, e.g., qPCR or hybridization arrays.

Few genomic tests are currently available in the clinical laboratory setting, and few technical staff have experience in the isolation, purification and amplification of labile mRNA for technologies such as qPCR and microarray. Use of molecular diagnostic technologies can provide for standardized methods for tissue collection that preserve the integrity of the biological macromolecules (DNA, RNA, protein) with the cells, allowing for more accurate detection.

“Breast cancer behavior,” as used herein, means, for example, whether the breast cancer will result in an increased likelihood of recurrence of the breast cancer, whether the human has increased likelihood of survival or death and a selection of a course of treatment for the breast cancer.

Alterations in gene expression (overexpression or underexpression) of at least one member selected from the group consisting of RERG, LIV-1, CA XII, ERBB4 and BCL-2α can be employed in the methods described herein. In addition, alterations in gene expression (overexpression or underexpression) of at least one member selected from the group consisting of CD34, EDG1, NQO-1, PTGDS and SDF-1 can be employed in the methods described herein.

Humans whose treatment is optimized by the methods described herein can have an estrogen-receptor positive breast cancer that is a primary estrogen-receptor positive breast cancer (i.e., cancer arising from breast tissue, such as epithelial tissue) or a secondary estrogen-receptor positive breast cancer (i.e., cancer arising from an organ other than breast tissue that metastases to breast tissue).

The breast tissue sample employed in the methods described herein can include homogenates of breast cancer biopsies, which include populations of different cell types (e.g., epithelial, stromal, smooth muscle).

The breast cancer tissue sample can be from a pre-menopausal human or a post-menopausal human.

The breast cancer tissue sample employed in the methods of the invention can be a breast cancer tissue sample, such as a primary breast cancer tissue sample, from a human that is lymph node negative (i.e., the breast cancer has not spread to the lymph node) and the breast cancer is estrogen receptor positive; or can be a breast cancer tissue sample from a human that is lymph node positive breast cancer (i.e., the breast cancer has spread to the lymph node) and the breast cancer is estrogen receptor positive.

The breast cancer tissue sample can be from a human with stage 1 (I), 2 (II), 3 (III) or 4 (IV) estrogen-receptor breast cancer or a human with stage 1, 2, 3 or 4 estrogen-receptor positive and progesterone-receptor positive breast cancer.

The American Joint Committee on Cancer (AJCC) staging of breast cancer is based on a scale of 0-4, with 0 having the best prognosis and 4 having the worst. There are multiple sub-classifications within each Stage classification (Robbins and Cotran, Pathological Basis of Disease, 7^{th} ed., Kumar, V., et al. (eds), Elsevier Saunders (2005)). Patients that present with ductal carcinoma in situ (DCIS) or lobular carcinoma in situ (LCIS) are considered stage 0. An invasive carcinoma of less than about 2 cm in the greatest dimension and no lymph node involvement is considered Stage I. An invasive carcinoma of less than about 5 cm in the greatest dimension and about 1 to about 3 positive lymph nodes is considered Stage II. Stage III refers to an invasive carcinoma of less than about 5 cm in the greatest dimension and four or more axillary lymph nodes involved or to an invasive carcinoma no greater than about 5 cm in the greatest dimension with nodal involvement or to an invasive carcinoma with at least about 10 axillary lymph nodes involved or invasive carcinoma with involvement of ipsilateral internal lymph nodes or invasive carcinoma with skin involvement, chest wall fixation or inflammatory carcinoma. Stage IV refers to a breast carcinoma with distant metastases (Robbins and Cotran Pathological Basis of Disease, 7^{th} Edition, eds. V. Kumar, et al., A. K. Abbas and N. Fausto, Elsevier Saunders (2005)).

Clinical staging of breast cancer is an estimate of the extent of the cancer based on the results of a physical exam, imaging tests (e.g., x-rays, CT scans) and often biopsies of affected areas. Blood tests can also be used in staging.

Pathological staging can be done on patients who have had surgery to remove or explore the extent of the cancer, which can be combined with clinical staging (e.g., physical exam, imaging tests). In some cases, the pathological stage may be different from the clinical stage. For example, surgery may reveal that the cancer has spread beyond that predicted from a clinical exam.

In one embodiment, the breast tissue sample is a laser capture microdissection (LCM) breast tissue sample. LCM is known in the art and is described herein infra. LCM can result in collections of varying cell types (e.g., epithelial, stromal, smooth muscle) in varying numbers, such as 100 cells, 1000 cells, 2000 cells or 5000 cells. LCM can be employed to prepare a breast tissue sample that includes relatively pure populations of a single cell type, such as an epithelial cell, a stroma cell or a smooth muscle cell.

In another embodiment, the breast tissue sample is an intact tissue section breast tissue sample. Intact tissue section can be prepared employing established techniques. For example, an intact tissue section can be prepared by freezing a breast tissue sample obtained from a biopsy in O.C.T. (Optimum Cutting Temperature) and cryo-sectioning the intact breast tissue sample. The frozen intact tissue section is then placed on a glass slide and stained with hematoxylin and eosin to assess structural integrity. Additional frozen intact tissue sections are prepared for total RNA extraction, purification and analyzed by quantitative polymerase chain reaction (qPCR), as described infra.

Expression of the genes can be identified by detecting mRNA for the genes or the protein product of the gene (see, for example, U.S. Patent Application Nos. US 2005/0095607, US 2005/0100933 and US 2005/0208500, the teachings of all of which are hereby incorporated by reference in their entirety). Expression of the genes described herein can be assessed by measuring the messenger RNA (mRNA) of the gene in the breast cancer sample. The mRNA encoded by the genes and the gene product described herein (see, for example, Table 1). Techniques to identify mRNA are known in the art and include, for example, qPCR, as described infra.

Expression of the genes in the methods described herein can be assessed by amplifying a nucleic acid sequence of the gene and detecting the amplified nucleic acid by well-established methods, such as the polymerase chain reaction (PCR), including quantitative PCR (qPCR), reverse transcription PCR (RT-PCR), and real-time PCR (including as a means of measuring the initial amounts of mRNA copies for each sequence in a sample), real-time RT-PCR or real-time Q-PCR. Exemplary techniques to employ such detection methods would include the use of one or two primers that are complementary to portions of a gene of interest (See Table 1), where the primers are used to prime nucleic acid synthesis. The newly synthesized nucleic acids are optionally labeled and may be detected directly or by hybridization to a gene or mRNA. The newly synthesized nucleic acids may be contacted with polynucleotides of a breast tissue sample under conditions which allow for their hybridization. Additional methods to detect the expression of genes in the methods described herein include RNAse protection assays, including liquid phase hybridizations and in situ hybridization of cells.

The breast tissue sample can be a biopsy sample that includes at least one member selected from the group consisting of breast epithelial cells, breast stromal cells, breast smooth muscle cells, which can include breast cancer cells of these tissue types. The breast tissue sample can be a breast biopsy that includes a carcinoma (ductal, lobular, medullary and/or tubular carcinoma). The breast tissue sample can be a breast biopsy that includes stroma. The breast tissue sample can be subjected to laser capture microdissection (LCM) in which relatively pure populations of carcinoma cells (cancerous cells of breast epithelium) and/or relatively pure populations of stromal cells are obtained. “Relatively pure,” as used herein in reference to a carcinoma or stromal breast tissue sample, means that the sample is about 95%, about 98%, about 99% or about 100% one cell type (e.g., carcinoma or stroma).

The methods described herein may be used in combination with other methods of diagnosing breast cancer to thereby more accurately identify a mammal at an increased risk for recurrence of breast cancer. For example, the methods described herein may be employed in combination or in tandem with assessments of the presence or absence of Ki-67, an antigen that is present in all stages of the cell cycle except GO and can be employed as a marker for tumor cell proliferation, and prognostic markers (including oncogenes, tumor suppressor genes, and angiogenesis markers) like p53, p27, Cathepsin D, pS2, multi-drug resistance (MDR) gene, and CD31. Alone or in combination with other clinical correlates of breast cancer, the methods described here may increase the accuracy of detection of breast cancer, in particular, in mammals who have had at least one or more incidents of breast cancer, thereby optimizing treatment of the breast cancer to decrease likelihood of recurrence of the breast cancer.

Increases (up-regulation of expression) and decreases (down-regulation of expression) of genes in the method described herein may be expressed in the form of a ratio between expression in a cancerous breast cell or a Universal Human Reference RNA (Stratagene, La Jolla, Calif.) (also referred to herein as a “control”) (See, for example, Table 1). For example, a gene can be considered up-regulated if the median expression value relative to a control, such as a Universal Human Reference RNA, is above one (1) (See, for example, Table 1). Likewise, a gene can be considered down-regulated if the median expression value relative to a control, such as a Universal Human Reference RNA, is less than one (1) (See, for example, Table 1).

Expression levels can be readily determined by quantitative methods as described herein, such as nucleic acid amplification assays. The methods described herein can identify over-expression (increases) or under-expression (decreases) of genes of Table 1 compared to a Universal Human reference RNA control. Over-expression or under-expression can be correlated with patient characteristics (e.g., age, menopausal stage, disease-free) and breast cancer characteristics (e.g., grade stage, estrogen receptor status, progesterone receptor status).

Over and under expression of genes described herein can be assessed by determining the Hazard Ratio (HR) by the methods described herein. HR less than one (1) indicates that the gene is overexpression and HR over one (1) indicates that the gene is underexpressed.

Expression of the genes described herein can be assessed as a ratio of the expression of the gene in a breast tissue sample from the mammal and a control tissue sample, such as from another mammal with breast cancer, from a sample of the same mammal from a previous breast cancer incident, or a mammal without breast cancer (also referred to herein as “normal” or “non-cancerous”). For example, an increase in the ratio of expression of the gene in the breast tissue sample from the mammal compared to a non-cancerous sample, may indicate an increased likelihood of recurrence of the breast cancer. The ratios of increased expression can be about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 30, about 40, about 50, about 60, about 70, about 80, about 90, about 100, about 150, about 200, about 300, about 400, about 500, about 600, about 700, about 800, about 900 or about 1000. For example, a ratio of 2 is a 100% (or a two-fold) increase in expression. Likewise, a decrease in gene expression can be indicated by ratios of about 0.9, about 0.8, about 0.7, about 0.6, about 0.5, about 0.4, about 0.3, about 0.2, about 0.1, about 0.05, about 0.01, about 0.005, about 0.001, about 0.0005, about 0.0001, about 0.00005, about 0.00001, about 0.000005 or about 0.000001, which may indicate a decreased likelihood of recurrence of breast cancer in the mammal.

Similarly, increases and decreases in expression of the genes described herein can be expressed based upon percent or fold changes over expression in non-cancerous cells. Increases can be, for example, about 10, about 20, about 30, about 40, about 50, about 60, about 70, about 80, about 90, about 100, about 120, about 140, about 160, about 180 or about 200% relative to expression levels in non-cancerous cells. Alternatively, fold increases may be of about 1, about 1.5, about 2, about 2.5, about 3, about 3.5, about 4, about 4.5, about 5, about 5.5, about 6, about 6.5, about 7, about 7.5, about 8, about 8.5, about 9, about 9.5 or about 10 fold over expression levels in non-cancerous cells. Likewise, decreases may be of about 10, about 20, about 30, about 40, about 50, about 55, about 60, about 65, about 70, about 75, about 80, about 85, about 90, about 95, about 98, about 99 or 100% relative to expression levels in non-cancerous cells.

Exemplary methods to assess relative gene expression analyses include employing the ΔΔCt method, in which the threshold cycle number (C_{T} value) is the cycle of amplification at which the qPCR instrument system recognizes an increase in the signal (e.g., SYBR® green florescence) associated with the exponential increase of the PCR product during the log-linear phase of nucleic acid amplification. These C_{T} values are compared to those of a housekeeping gene, such as glyceraldehyde phosphate dehydrogenase (GAPDH) or β-actin to obtain the ΔCt value, which is used to normalize for variation in the amount of RNA between different samples. The ΔCt value of each gene is then compared to that present in a calibrator, such as Universal Human Reference RNA (Stratagene, La Jolla, Calif.), in order to obtain a ΔΔCt value. Since each cycle of amplification doubles the amount of PCR product, the expression level of a target gene relative to that of the calibrator is calculated from 2^{-ΔΔCt}, expressed as relative gene expression.

In an additional embodiment, the invention is an immobilized collection (microarray) of the genes, such as a gene chip, described herein (Table 1) for ease of processing in the methods described herein. The gene chips that include the genes described herein can permit high throughput screening of numerous breast tissue samples. The genes identified in the methods described herein can be chemically attached to locations on an immobilized collection, such as a coated quartz surface. Nucleic acids from breast tissue samples can be prepared as described herein and hybridized to the genes and expression of the genes identified.

In another embodiment, the invention includes kits to perform the methods described herein.

The teachings of all patents, published applications and references cited herein are incorporated by reference in their entirety.

### Exemplification

Previous studies have described subtypes of breast cancer with specific molecular signatures (e.g., distinct microarray profiles), which appear to represent a range of breast carcinomas exhibiting less aggressive clinical phenotypes to those with very aggressive behavior as indicated by the Kaplan Meier analyses of disease-free survival (1, 2). These date suggest that expression of certain genes in each subtype (e.g., Stage 1, 2, 3 or 4 of breast cancer) is associated with an apparent, yet unexplained, effect on prognosis. While there was a distinction among each subtype based on survival analysis, the gene expression data were obtained from microarray profiling, which is subject to a variety of variables, and the magnitude of the genes in each “signature” ranged from about 100 to about 200 genes. The method described herein detects the expression (up-regulated or down regulated) of a relatively small number of genes to predict the increased likelihood of estrogen receptor breast cancer in a mammal and the likelihood of survival if the breast cancer reoccurs. The invention described herein can be employed to assess the likelihood of response to drugs that perturb the signal signaling of estrogen receptor (ER), such as compounds that bind to the ER (e.g., Tamoxifen™ (Nolvadex®), Raloxifene®), which, in turn, can impact clinical management of breast cancer treatment.

Using pure populations of laser captured breast cancer cells, statistically significant subsets of ER-associated genes from altered expression can either be found in subtype A (e.g, RERG, LIV-1, CAXII, ERBB4, BCL2) (also referred to herein as “A molecular sybtype” or “A subtype”) or in subtype B (e.g., CD34, EDG-1, NQO-1, PTDGS, SDF-1) (also referred to herein as “B molecular subtype” or “B subtype”) breast cancer biopsies.

Gene Expression Analyses by qPCR

Total RNA was purified from frozen tissue specimens using RNeasy™ reagents (Qiagen) per manufacturer protocol, incorporating an on-column DNAse digestion step. Isolated RNA (200-500 ng in 10 μL) was then combined with 166 ng random hexamers (Promega) 10 nmoles of dNTPs (Invitrogen) and H₂O for a total volume of 13 μL. Primer annealing was performed at 65° C. for 5 minutes, followed by addition of enzyme mix containing 1 μL SuperScript™ III reverse transcriptase with 4 μL 2× Master Mix (Invitrogen), 1 μL RNAsin™ RNAse inhibitor (Promega), and 1 μL dithiothreitol for a total volume of 20 μL. Each reaction was incubated at 25° C. for 5 minutes, followed by 55° C. for 60 minutes, then 75° C. for 15 minutes. cDNA was stored at −20° C.

Real-time PCR was performed using SYBR™ Green detection (Applied Biosystems). cDNA was diluted 20-fold in 2 ng/μl polyinositol (Sigma) and combined with 2× SYBR™ Green master mix to each 12 μL reaction with forward and reverse primers (300 nM final concentration). Primers were selected using each gene sequence as a template for the Primer Express™ (Applied Biosystems) PCR primer selection tool. PCR was performed using 40 cycles (58° C. for 30 seconds, 72° C. for 30 seconds then 95° C. for 15 seconds). Primers for PCR reactions for each gene are listed in Table 1. For each experiment, reactions were completed in duplicate wells using the 7900HT Sequence Detection System (Applied Biosystems). Using β-actin as the reference gene, relative expression levels of each target gene were determined by the ΔΔCt method (Pfaffl. M. W. Nucleic Acids Res 29:e45 (2001)). Human Universal Reference RNA (Stratagene) was analyzed by qPCR in parallel (500 ng per reverse transcription reaction) for use as the calibrator in the ΔΔCt calculation. Experiments were performed in triplicate to calculate a mean±standard deviation. Both no template and RNA (no RT) reactions were used as negative controls.

Determination of expression (overexpression or underexpression) of genes listed in Table 1 can be employed in the methods of the invention to treat breast cancer (ER⁺/PR⁺) in a human.

TABLE 1

| | Unigene | Genbank | | |
|---|---|---|---|---|
| Gene | ID | Accession No. | Forward Primer | Reverse Primer |
| ESR1 | Hs.208124 | NM_000125 | GCCAAATTGTGTTTGATG | GACAAAACCGAGTCACATCAG |
| | | | GATTAA (SEQ ID NO: 1) | TAATAG (SEQ ID NO: 2) |
| PGR | Hs.32405 | NM_000926 | AGAGCACTGGATGCTGT | TGGCTTAGGGCTTGGCTTT |
| | | | TGCT (SEQ ID NO: 3) | (SEQ ID NO: 4) |
| EGFR | Hs.488293 | NM_005228 | GCACATTTTGGGAAGTTG | ATTTCTGCTCAAAGGGACAAT |
| | | | CAT (SEQ ID NO: 5) | ATTCT (SEQ ID NO: 6) |
| HER2 | Hs.446352 | NM_004448 | AAAAGCGACCCATTCAGA | AAAAACTAAACAGAAAAGCAC |
| | | | GACT (SEQ ID NO: 7) | TCTGTACAA |
| | | | | (SEQ ID NO: 8) |
| BCL2a | Hs.150749 | NM_000633.2 | GCCCCAAAAGGAGAAGA | TTCTGCCCCTGCCAAATCT |
| | | | ACATC (SEQ ID NO: 9) | (SEQ ID NO: 10) |
| CAXII | Hs.210995 | NM_001218 | CAGGCGCAACTCCTCCA | GGTCGGTTCCTTCTCAGTCAT |
| | | | TT (SEQ ID NO: 11) | G (SEQ ID NO: 12) |
| ERBB4 | Hs.390729 | Non-specific, | GTCCTGTACTGGCCGTT | AGCATCTGCCGTCACATTGTT |
| total | | measures all | GCT (SEQ ID NO: 13) | (SEQ ID NO: 14) |
| | | variants | | |
| ERBB4 | Hs.390729 | NM_005235.2 | GGCCATTCCACTTTACCA | CAGAATGAAGAGCCCACCAAT |
| JM-a | | NM_001042599.1 | CAA (SEQ ID NO: 15) | T (SEQ ID NO: 16 |
| LIV1 | Hs.719277 | NM_012319 | GCAGGCTGTCCTTTATAA | TGAAAATTCCTGTTGCCATTC |
| | | | TGCA (SEQ ID NO: 17) | C (SEQ ID NO: 18) |
| RERG | Hs.199487 | NM_032918 | CTCCAGGCAGGTTAGCA | AGGCAGAGCACTCGTAAAAAG |
| | | | CAGA (SEQ ID NO: 19) | C (SEQ ID NO: 20) |
| CD34 | Hs.374990 | NM_001773 | CTCCAGAAACGGCCATT | CCCACCTAGCCGAGTCACAA |
| | | | CAG (SEQ ID NO: 21) | (SEQ ID NO: 22) |
| EDG1 | Hs.154210 | NM_001400 | CTCTTCTGCACCACGGTC | CTCCGAGTCCTGACCAAGGA |
| | | | TTC (SEQ ID NO: 23) | (SEQ ID NO: 24) |
| PTGDS | Hs.446429 | NM_000954 | AAATTCACCGCCTTCTGC | TGTTCCGTCATGCACTTATCG |
| | | | AA (SEQ ID NO: 25) | (SEQ ID NO: 26) |
| NQO1 | Hs.406515 | NM_000903 | GATTGGACCGAGCTGGA | CAGCCGTCAGCTATTGTGGAT |
| | | | AAAC (SEQ ID NO: 27) | A (SEQ ID NO: 28) |
| SDF1 | Hs.522891 | NM_000609 | GGGAAATATTCCCTAGAA | GAGTCCAGCGAGGTTGCAA |
| tv2 | | | ACTTCCA | (SEQ ID NO: 30) |
| | | | (SEQ ID NO: 29) | |

Characteristics of Patients and Disease with the A and B Molecular Subtypes

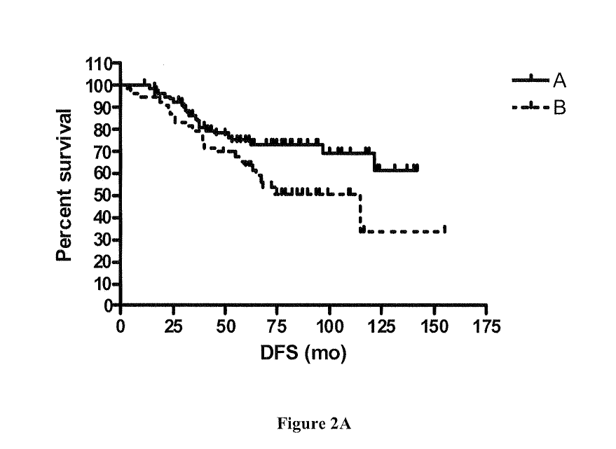

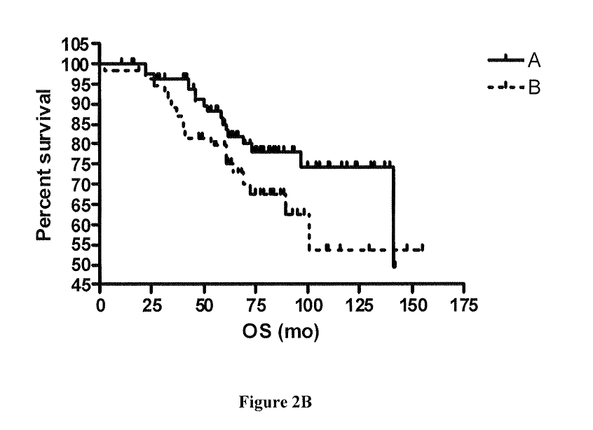

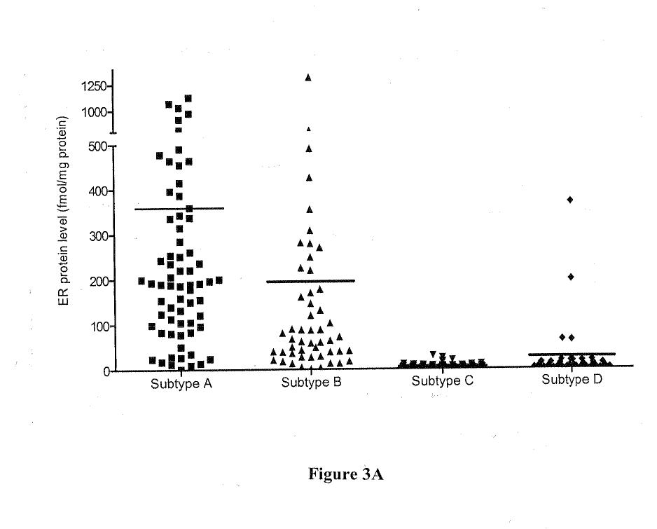

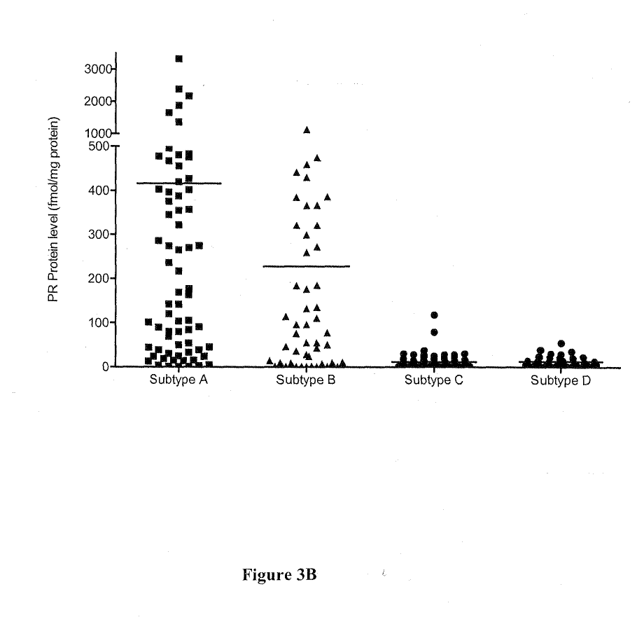

In order to insure that the clinical behaviors identified in the A and B molecular subtypes are related to gene expression and are not merely due to clinical differences in the population, certain clinical characteristics of breast cancer were examined. A higher percentage of node positive cancers in the B subtype relative to the cases in subtype A (46% vs 35% respectively) and a higher percentage of grade 3 cases in subtype B compared to biopsies exhibiting subtype A (32% vs 16% respectively) were observed. These clinical characteristics, however, do not explain the significant differences in survival probabilities (FIGS. 2A and 2B), especially considering there were relatively equal proportions of stages 1 and 2 cancers in each molecular subtype and a higher number of stage 4 cancers in the A subtype, which exhibited the better survival (FIGS. 2A and 2B). In addition, patients with breast cancers exhibiting the molecular subtype B were given a greater number of therapeutic treatments than subtype A, and yet their survival results were less than those of patients with molecular subtype A. Considering ER/PR status, most of the patients were both ER+/PR+ in the A and B molecular subtypes (87% and 73%, respectively), with only four ER− cases in subtype A, and five ER− cases in subtype B. Most of these cases were highly positive for ER and PR, although the quantitative expression data showed lower mean expression values for each biomarker in the molecular subtype B patients (FIGS. 3A and 3B). The levels of ER and PR detected in the biopsies are above the clinical cutoff values used in the management and treatment of breast cancer.

### Selection of Gene Subsets for Evaluation of Their Utility in the Clinical Management of Human Breast Carcinoma

Statistical analyses of expression levels of genes composing the two estrogen receptor positive (ER+) molecular subtypes A and B obtained from LCM-procured breast carcinoma cells from frozen human tissue biopsies revealed the fifty genes in each subtype that were significantly over-expressed (1, 2). These 100 genes were evaluated using ERTargetDB (3) to screen the promoter regions of each gene for candidate ERE sequences (Table 2). This database also contained literature-based information describing estrogen-dependent expression of these genes (Table 2). These analyses revealed ten candidate genes within the A and B subtypes that were associated with ER signaling (Table 2). Additional genes that represent conventional tumor markers for assessing prognosis and selecting endocrine therapy (e.g., Tamoxifen™) of breast cancer (ER and progestin receptor (PR)) were added to the panel. In addition, the expression of genes for both EGFR & HER-2 were also assessed. ERBB4 itself is an important member of the ERBB receptor family along with EGFR (ERBB1) and HER2 (ERBB2). The genes selected were based upon the probes used for the microarray analysis with one addition. The ERBB4 probe on the microarray platform was not specific for any of the splice variants. For expression of the 3M-a variant (7-10), qPCR primers specific for thJM-a variant were used in addition to primers designed to measure overall ERBB4 expression.

TABLE 2
Description of Gene Subset

| | | | | | |
|---|---|---|---|---|---|
| | | | | ERE | Estrogen- |
| Gene | UniGene ID | Description | Subtype | present | regulated |

ER-associated genes selected from A & B molecular subtypes

| | | | | | |
|---|---|---|---|---|---|
| BCL-2 α | Hs.150749 | B-cell CLL lymphoma 2 alpha | A | Yes | Yes |
| CA XII | Hs.210995 | Carbonic anhydrase XII | A | Imperfect | Yes |
| ERBB4 | Hs.390729 | Human epidermal growth factor | A | Half-site | Yes |
| | | receptor 4 | | | |
| LIV-1 | Hs.719277 | Estrogen-regulated zinc transporter | A | | Yes |
| RERG | Hs.199487 | RAS-like, estrogen-regulated | A | Yes | Yes |
| | | growth inhibitor | | | |
| CD34 | Hs.374990 | CD34 antigen | B | Yes | Yes |
| EDG-1 | Hs.154210 | Endothelial differentiation | B | Yes | Yes |
| | | sphingolipid GPCR 1 | | | |
| NQO-1 | Hs.406515 | NAD(P)H dehydrogenase quinine 1 | B | Yes | |
| PTGDS | Hs.446429 | Prostaglandin D2 synthase | B | Candidate | Yes |
| SDF-1 | Hs.522891 | Stromal cell-derived factor 1 | B | Half-site | Yes |

Genes in subset representing conventional tumor markers

| | | |
|---|---|---|
| ESR1 | Hs.208124 | Estrogen receptor alpha |
| PGR | Hs.32405 | Progestin receptor |
| EGFR | Hs.488293 | Human epidermal growth factor |
| | | receptor 1 |
| HER-2 | Hs.446352 | Human epidermal growth factor |
| | | receptor 2 |

The methods of the invention can include detecting the expression (e.g., up-regulation or down-regulation) on of the genes listed in Table 2, which can be important in selecting particular therapies.

Clinical Utility of the Expression Profile (Molecular Signature) of the ER-Associated Gene Subset in Biopsies from ER+ Primary Breast Cancers for Predicting a Patient's Risk of Recurrence

An independent population of ER-positive, early stage (i.e., stage 1 and stage 2) primary breast cancer biopsies were selected based on clinical and pathological features. The samples were obtained from pre- and postmenopausal women. RNA in the samples was analyzed by qPCR for assessment of expression levels of genes, in particular, the genes listed in Table 2. While many patients with these clinical and pathologic characteristics exhibit good prognosis (e.g., increased disease-free, disease free survival (DFS) and overall survival (OS)), others had breast cancer recurrences (metastases).

### Specimen Selection

Using the clinical data available in our IRB-approved Database, cases were selected matching the above described criteria, then evaluated for histologic confirmation and RNA quality control analyses before proceeding with qPCR. The clinical criteria included ER-positive status, stages 1 and 2, known lymph node status and a minimum event-free follow-up of 24 months. Table 3 lists the characteristics of this patient population. Currently, 210 cases passed the QC analyses and clinical selection criteria for analysis of both DFS and OS probabilities (never disease-free cases excluded from DFS analyses). Examination of accepted clinical parameters, e.g., stage, grade, nodal status, as a function of DFS and OS indicated the test population was representative of the general population. Disease-free survival (DFS) is defined as a time between the initial detection and removal of the primary lesion (mastectomy), if no other disease is present and the appearance of the first metastasis (i.e., recurrence), regardless of whether it is distant or local. Overall survival (OS) is defined as the time between the initial detection and removal of the primary lesion (mastectomy), if no other disease is present and death of the patient due to the presence of the cancer.

TABLE 3
Patient population for clinical utility of invention

| Patient Parameters | n |
|---|---|
| Median Age (range) | |
| 62 years (29-89.5) | 210 |
| Median Observation time (range) | |
| 78 months (3-154) | |
| Race | |
| white | 207 |
| black | 3 |
| Histology | |
| Invasive ductal Carcinoma | 180 |
| Invasive lobular Carcinoma | 24 |
| Other | 6 |
| Median Tumor Size (Range) | |
| 22 mm (6-85) | 202 |
| Stage | |
| 1 | 65 |
| 2A | 81 |
| 2B | 64 |
| Grade | |
| 1 | 27 |
| 2 | 79 |
| 3 | 47 |
| 4 | 1 |
| unknown | 56 |
| Lymph Node Status | |
| negative | 127 |
| positive | 83 |
| PR Status | |
| negative | 29 |
| positive | 181 |
| Recurrence Status | |
| yes | 46 |
| no | 152 |
| never disease-free | 12 |
| Adjuvant Therapy | |
| Tamoxifen ™ | 60 |
| Chemotherapy | 28 |
| Raditation | 19 |

Detection of Gene Expression by qPCR

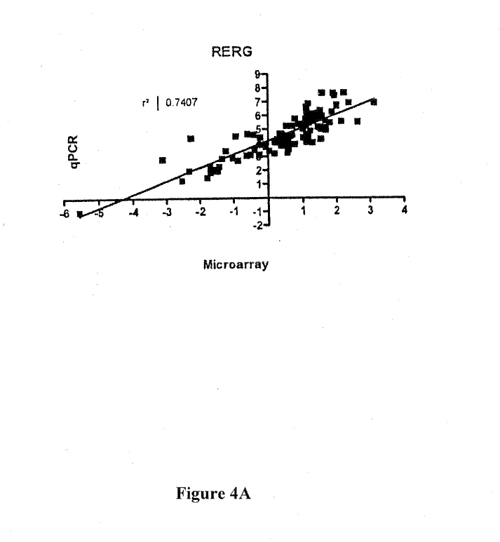

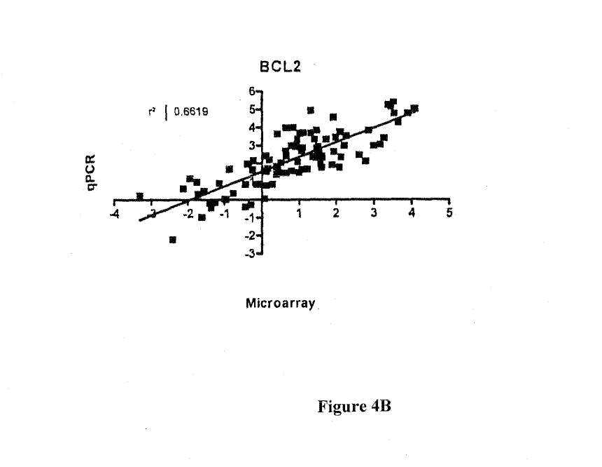

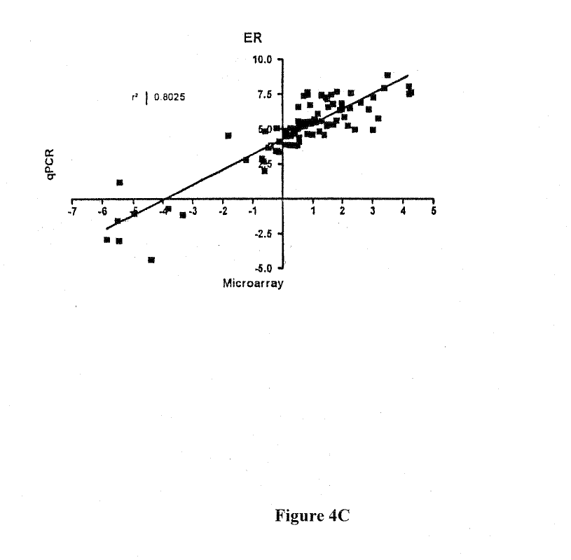

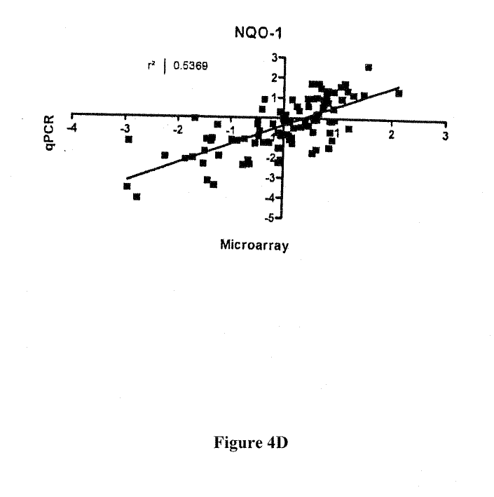

Previous, gene expression signatures were too large for experimentation and commercialization, being composed of 200 genes. Certain ER associated genes were statistically significantly expressed in only subtype A (e.g, RERG, LIV-1, CAXII, ERBB4, BCL2) or only in subtype B (e.g., CD34, EDG-1, NQO-1, PTDGS, SDF-1) breast cancers. These gene subsets were analyzed by qPCR in a new population of ER+ primary breast cancers. Linear regression analyses were performed comparing the different platforms (qPCR vs microarray, n=92) for expression determinations of 14 candidate genes. FIGS. 4A, 4B, 4C and 4D illustrate representative comparisons of four of the genes across platforms. Most of the genes analyzed exhibited positive linear relationships with the microarray data with strong correlation, indicated by R² values >0.5 (FIGS. 4A, 4B, 4C and 4D).

### Multivariate Analyses of Gene Expression

For each of the multi-gene models derived, Kaplan-Meier regression was performed using odds values calculated from the following equation (11):

Odds=e^{(B}₁^{X}₁^{+B}₂^{X}₂^{+ . . . +BnXn)}

B (beta) is a coefficient calculated for expression of each gene using the Cox proportional hazards model, and X is the log₂-transformed value of the relative gene expression determine by qPCR. Negative B values indicate that decreased expression is associated with decreased survival, which also corresponds to hazard ratios <1. Lower odds values, which are calculated for each patient, are associated with decreased risk of recurrence/mortality. Odds ratios may also be determined by using the odds value calculated from the median expression of each gene in the model, and normalizing all odds values to that number.

For the categorical Kaplan-Meier regression analyses using the multivariate models, each population was initially divided into thirds (low, intermediate and high risk) according to the odds value calculated for each patient's specimen. For each analysis, the low and intermediate groups exhibited similar survival probabilities, and were subsequently combined to represent a low-risk group containing two-thirds of the population, and a high-risk group consisting of the upper one-third of the odds values obtained from the corresponding model. Due to the smaller population of node-positive and Tamoxifen™-treated patients (n<100), they were divided equally into low and high risk groups.

Gene Subsets in Patients with Stages 1-2 Breast Cancers Exhibiting ER+ Tissue Biopsies without Regard to PR Status

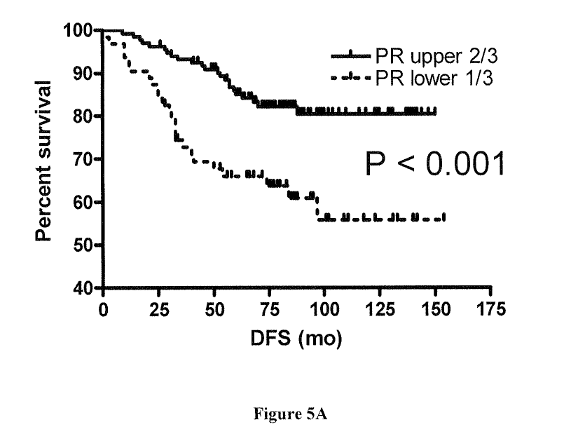

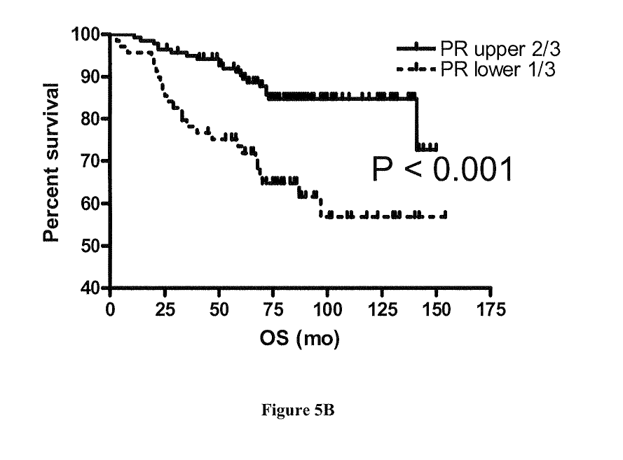

Univariate Cox regression analyses revealed that the expression of five genes independently correlated (P<0.05) with both disease-free (DFS) and overall (OS) survival (Table 4). These include four genes from subtype A (BCL2, CA XII, ERBB4 JM-a and RERG) and PR. Multivariate Cox regression was performed using both forward and backward conditional selection methods, and there were no multi-gene models obtained that achieved equivalent significance as that achieved by assessing PR expression (Table 5). FIGS. 5A and 5B illustrate Kaplan-Meier analyses of DFS (FIG. 5A) and OS (FIG. 5B) as a function of PR gene expression. As shown, increased PR expression in a breast cancer biopsy was associated with an about a 25% increase in both disease-free and overall survival probabilities.

These data indicate that while ER is considered by many as the major prognostic marker and therapeutic target for treating breast cancer, PR also exhibits a very strong clinical relevance in ER+ breast cancers. This reflects not only its utility for correctly assessing a patient's prognosis, but also its potential as a molecular target for design of new drugs. Due to the significance of PGR expression, more focused analyses were performed on primary breast cancers positive for both ER and PR. Patients with breast cancers positive for both steroid receptors are considered to have better prognosis (low risk of recurrence) and better response to hormone therapy (i.e., Tamoxifen™, aromatase inhibitors) than those positive for only one of these two tumor markers (12). The methods of the invention include determining the expression of PGR, BCL2a, CAXII, ERB B4 JM-a and RERG in a breast cancer sample.

TABLE 4
Univariate Cox Regression Survival Analyses in ER+, Stage 1-2
Population

| | DFS analysis | | OS analysis | |
|---|---|---|---|---|
| | (n = 208) | | (n = 208) | |
| Gene | Exp (B) = HR | P value | Exp (B) = HR | P value |
| ER | 0.92 | 0.09 | 0.92 | 0.11 |
| PR | 0.86 | 0.001 | 0.86 | 0.002 |
| EGFR | 1.00 | 0.97 | 0.96 | 0.72 |
| HER2 | 1.02 | 0.84 | 1.06 | 0.54 |
| BCL2a | 0.83 | 0.04 | 0.82 | 0.03 |
| CAXII | 0.80 | 0.001 | 0.82 | 0.002 |
| ERBB4 total | 0.91 | 0.06 | 0.91 | 0.06 |
| ERBB4 JM-a | 0.87 | 0.02 | 0.87 | 0.02 |
| LIV1 | 0.86 | 0.07 | 0.93 | 0.41 |
| RERG | 0.80 | 0.004 | 0.83 | 0.02 |
| CD34 | 0.88 | 0.40 | 0.89 | 0.45 |
| EDG1 | 0.90 | 0.42 | 0.95 | 0.73 |
| NQO1 | 0.84 | 0.12 | 0.94 | 0.58 |
| PTGDS | 1.02 | 0.84 | 0.97 | 0.71 |
| SDF1tv2 | 0.89 | 0.35 | 0.86 | 0.26 |

TABLE 5
Multivariate Cox Regression Analyses in ER+, Stage 1-2 Population

| DFS model | | | OS model | | |
|---|---|---|---|---|---|
| Gene | Exp (B) = HR | P value | Gene | Exp (B) = HR | P value |
| PR | 0.86 | 0.002 | PR | 0.86 | 0.002 |

Expression of a Gene Subset in Tissue Biopsies from Patients with ER+/PR+ Breast Cancers

Univariate Cox regression revealed the expression of three genes, CA XII, ERBB4 and RERG, significantly correlated with DFS and OS, while PGR only had a significant association with OS. It should be noted that independent assessment of the JM-a splice variant exhibited greater significance than measurement of total ERBB4 expression. Multivariate analyses revealed a four gene model (HER-2, CA XII, ERBB4 JM-a and LIV-1) for DFS, with the same genes incorporated into the most significant OS model (Table 7). HER-2 expression exhibited the expected negative impact on survival (HR=1.30 & 1.27 for DFS and OS, respectively). Although it appeared in Subtype A, LIV-1 expression also exhibited a negative impact on survival (HR=1.31 & 1.39, respectively). This indicates that interactions among genes within this subset may affect the clinical impact of their expression.

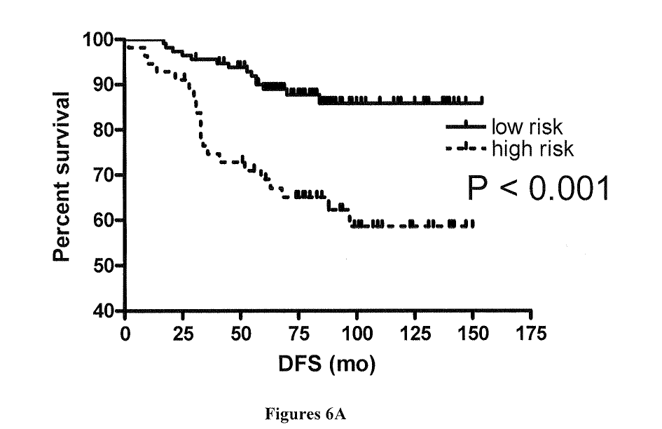

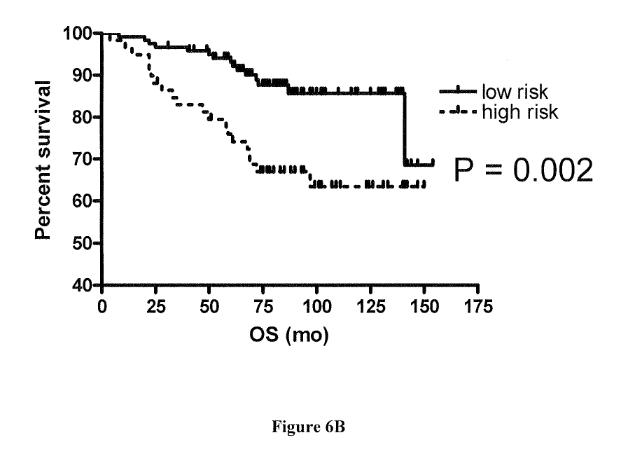

Using the four gene models, Kaplan-Meier regression analyses were employed to stratify the patients within this group according to odds values determined from the equation mentioned previously. As shown in FIGS. 6A and 6B, the high risk DFS group (n=59) exhibited about 30% greater recurrence after 10 years than the low risk group (n=120). Similar separation was obtained in the OS analysis, except for one patient that died of disease at 141 months. Since most of the cases were censored at the end of their follow-up by 140 months, this single mortality created a significant drop in the survival curve because the remaining uncensored data had a total n value of 5 and one mortality represents about 20% of the remaining dataset. Overall, the high and low risk groups exhibited very significant separation for both DFS and OS analyses (P<0.001 and P=0.002 respectively).

TABLE 6
Univariate Cox Regression Survival Analyses in ER+/PR+, Stage 1-2
Population

| | DFS analysis | | OS analysis | |
|---|---|---|---|---|
| | (n = 179) | | (179) | |
| Gene | Exp (B) = HR | P value | Exp (B) = HR | P value |
| ER | 0.92 | 0.23 | 0.91 | 0.17 |
| PR | 0.89 | 0.06 | 0.85 | 0.009 |
| EGFR | 0.99 | 0.96 | 1.02 | 0.88 |
| HER2 | 1.19 | 0.13 | 1.15 | 0.23 |
| BCL2a | 0.84 | 0.12 | 0.82 | 0.07 |
| CAXII | 0.76 | 0.01 | 0.78 | 0.02 |
| ERBB4 total | 0.90 | 0.05 | 0.90 | 0.05 |
| ERBB4 JM-a | 0.81 | 0.01 | 0.80 | 0.009 |
| LIV1 | 0.92 | 0.39 | 0.95 | 0.62 |
| RERG | 0.75 | 0.02 | 0.76 | 0.02 |
| CD34 | 0.84 | 0.32 | 0.91 | 0.59 |
| EDG1 | 0.83 | 0.25 | 0.91 | 0.57 |
| NQO1 | 0.84 | 0.21 | 0.94 | 0.65 |
| PTGDS | 0.98 | 0.87 | 0.94 | 0.52 |
| SDF1tv2 | 1.03 | 0.86 | 0.95 | 0.73 |

TABLE 7
Multivariate Cox Regression Analyses in ER+/PR+, Stage 1-2
Population

| DFS model | | | OS model | | |
|---|---|---|---|---|---|
| | Exp (B) = | | | Exp (B) = | |
| Gene | HR | P value | Gene | HR | P value |
| HER2 | 1.30 | 0.008 | HER2 | 1.27 | 0.02 |
| CA XII | 0.64 | 0.02 | CA XII | 0.63 | 0.02 |
| ERBB4 | 0.81 | 0.06 | ERBB4 JM-a | 0.78 | 0.03 |
| JM-a | | | | | |
| LIV1 | 1.31 | 0.08 | LIV1 | 1.39 | 0.04 |

Patient Population with ER+/PR+, Node-Negative Breast Cancers

Nodal status is an important prognostic indicator for breast cancer. Patients in the hormone receptor positive population were divided according to nodal status to determine the influence of expression of the gene subset in node positive (breast cancer detected in nodal biopsies) and node negative (breast cancer not detected in nodal biopsies). In the node-negative population (Table 8), no single gene independently correlated with DFS (n=111) nor with OS (n=110); however, the multivariate analyses revealed a five-gene model for DFS and a six-gene model for OS (Table 9). There are four genes common to each of these models CA XII, ERBB4 total, CD34 and EDG1, with the DFS model incorporating ESR1 and the OS model incorporating LIV1 and NQO1. Total expression of ERBB4 was measured, rather than specific JM-a expression. In the methods described herein, expression of both ERBB4 and JM-a can be determined, the prognostic significance is population-dependent. ER exhibits a negative prognostic impact in the DFS analysis (HR=1.41). Interactions among genes and their pathways may influence the clinical significance in predicting breast cancer behavior. Even an accepted biomarker, such as ER, may have its biological activity influenced by other genes in a manner that alters its effect on cancer progression in certain populations.

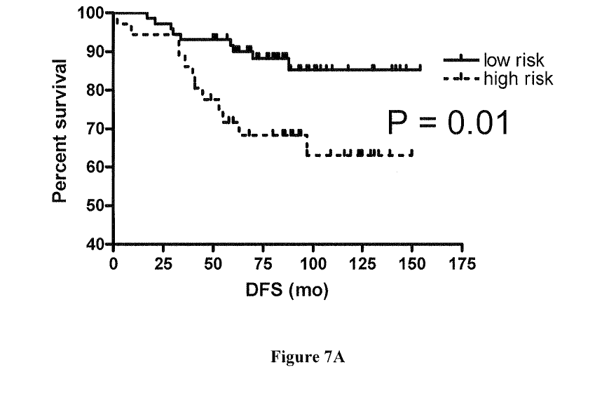

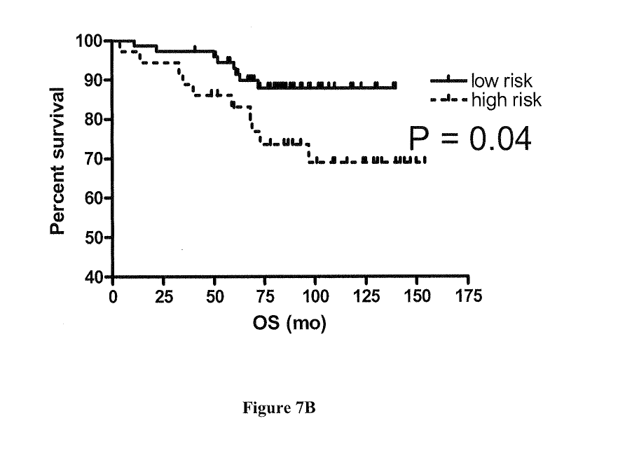

FIGS. 7A and 7B illustrate Kaplan-Meier regression analyses of both DFS and OS in the ER+/PR+, node-negative population. About a 25% increase in recurrence probability in the high risk group (n=37) compared to that of the low risk group (n=74). Likewise, similar separation was noted in the OS analysis (about 20%) between high risk (n=36) and low risk (n=74) groups. Given that this population of hormone receptor positive, node-negative cancers represents patients likely to exhibit the best prognostic behavior for invasive breast cancers, the ability to classify a group with about 40% recurrence and about 35% mortality rates strongly suggests this gene subset is an extremely powerful tool for detecting high risk breast cancers that would otherwise be determined as low risk using conventional clinical and pathologic features.

TABLE 8
Univariate Cox Regression Survival Analyses in ER+/PR+, Node-
negative Population

| | DFS analysis | | OS analysis | |
|---|---|---|---|---|
| | (n = 111) | | (n = 110) | |
| Gene | Exp (B) = HR | P value | Exp (B) = HR | P value |
| ER | 1.05 | 0.66 | 1.08 | 0.57 |
| PR | 0.90 | 0.17 | 0.86 | 0.07 |
| EGFR | 0.95 | 0.72 | 0.97 | 0.87 |
| HER2 | 1.22 | 0.17 | 1.23 | 0.18 |
| BCL2a | 0.97 | 0.87 | 1.03 | 0.87 |
| CAXII | 0.90 | 0.51 | 0.89 | 0.52 |
| ERBB4 total | 0.87 | 0.26 | 0.84 | 0.16 |
| ERBB4 JM-a | 0.90 | 0.34 | 0.86 | 0.22 |
| LIV1 | 1.04 | 0.79 | 1.12 | 0.42 |
| RERG | 0.86 | 0.36 | 0.90 | 0.55 |
| CD34 | 1.02 | 0.92 | 1.21 | 0.41 |
| EDG1 | 0.85 | 0.40 | 1.04 | 0.85 |
| NQO1 | 1.19 | 0.34 | 1.43 | 0.08 |
| PTGDS | 0.90 | 0.40 | 0.95 | 0.71 |
| SDF1tv2 | 0.88 | 0.47 | 0.98 | 0.93 |

TABLE 9
Multivariate Cox Regression Analyses in ER+/PR+, Node-negative
Population

| DFS model | | | OS model | | |
|---|---|---|---|---|---|
| | Exp (B) = | | | Exp (B) = | |
| Gene | HR | P value | Gene | HR | P value |
| ER | 1.41 | 0.03 | CA XII | 0.51 | 0.02 |
| CA XII | 0.62 | 0.06 | ERBB4 total | 0.72 | 0.03 |
| ERBB4 total | 0.77 | 0.09 | LIV1 | 1.68 | 0.03 |
| CD34 | 2.42 | 0.04 | CD34 | 2.19 | 0.08 |
| EDG1 | 0.40 | 0.03 | EDG1 | 0.50 | 0.10 |
| | | | NQO1 | 1.63 | 0.04 |

### Expression of Gene Subsets in ER+/PR+, Node-Positive Breast Cancers

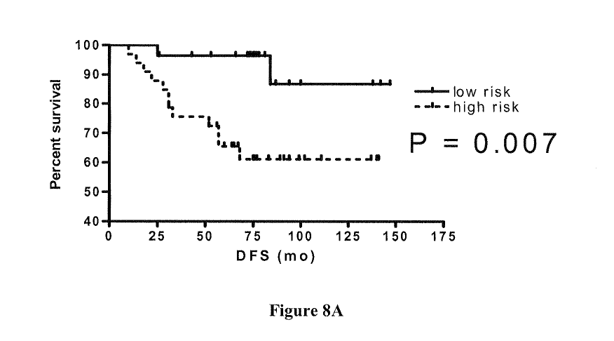

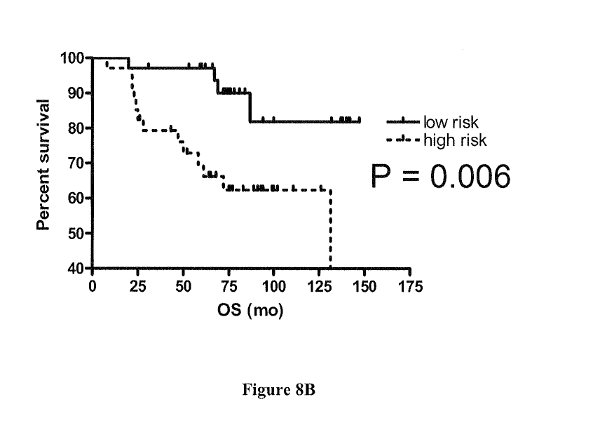

Univariate Cox regression analyses (Table 10) revealed six genes that independently correlated with DFS (n=68) and seven genes that correlated with OS (n=69). The multivariate analyses shown in Table 11 revealed a six gene model for DFS (HER2, BCL2a, CA XII, CD34, EDG1 and NQO1) and a three gene model for OS (ERBB4 JM-a, CD34 and EDG1). The Kaplan-Meier regression in FIGS. 8A and 8B illustrate a significant separation between risk groups for both DFS (P=0.007) and OS (P=0,006). Patients in the low risk DFS group (n=34) exhibited two recurrences, and patients in the low risk OS groups (n=34) exhibited four mortalities. These low risk groups represent subpopulations of node-positive patients that exhibit survival probabilities similar to those expected in node-negative patients.

Comparing the node-negative and node-positive survival curves, it is evident that the multi-gene models successfully classify groups of low and high risk patients for both DFS and OS that exhibit similar survival probabilities regardless of nodal status. This indicates the power of this gene subset in conjunction with accepted clinical parameters (e.g., nodal status) for assessment of prognosis (risk of recurrence) of hormone receptor positive breast cancers.

TABLE 10
Univariate Cox Regression Survival Analyses in ER+/PR+, Node-
positive Population

| | DFS analysis | | OS analysis | |
|---|---|---|---|---|
| | (n = 68) | | (n = 69) | |
| Gene | Exp (B) = HR | P value | Exp (B) = HR | P value |
| ER | 0.81 | 0.04 | 0.82 | 0.04 |
| PR | 0.88 | 0.25 | 0.85 | 0.09 |
| EGFR | 1.11 | 0.69 | 1.08 | 0.74 |
| HER2 | 1.16 | 0.43 | 1.12 | 0.54 |
| BCL2a | 0.73 | 0.06 | 0.73 | 0.03 |
| CAXII | 0.66 | 0.006 | 0.70 | 0.02 |
| ERBB4 total | 0.91 | 0.18 | 0.92 | 0.28 |
| ERBB4 JM-a | 0.66 | 0.008 | 0.70 | 0.02 |
| LIV1 | 0.78 | 0.15 | 0.83 | 0.24 |
| RERG | 0.60 | 0.02 | 0.68 | 0.03 |
| CD34 | 0.45 | 0.04 | 0.50 | 0.04 |
| EDG1 | 0.79 | 0.39 | 0.77 | 0.32 |
| NQO1 | 0.48 | 0.002 | 0.71 | 0.04 |
| PTGDS | 1.14 | 0.44 | 0.92 | 0.63 |
| SDF1tv2 | 1.03 | 0.92 | 0.79 | 0.36 |

TABLE 11
Multivariate Cox Regression Analyses in ER+/PR+, Node-positive
Population

| DFS model | | | OS model | | |
|---|---|---|---|---|---|
| | Exp (B) = | | | Exp (B) = | |
| Gene | HR | P value | Gene | HR | P value |
| HER2 | 1.51 | 0.02 | ERBB4 JM-a | 0.72 | 0.03 |
| BCL2a | 1.94 | 0.10 | CD34 | 0.24 | 0.02 |
| CA XII | 0.43 | 0.02 | EDG1 | 2.21 | 0.10 |
| CD34 | 0.14 | 0.03 | | | |
| EDG1 | 2.52 | 0.07 | | | |
| NQO1 | 0.50 | 0.01 | | | |

Expression of a Gene Subset in ER-Positive Breast Cancer Biopsies from Patients Treated with Tamoxifen™

In order to determine the relationship between expression of a gene subset and Tamoxifen™ response, expression of genes were identified in 60 patients from the ER+/PR+, stage 1-2 group who were treated with adjuvant Tamoxifen™ therapy. ER is the molecular target for Tamoxifen™ and patients with hormone receptor positive breast cancer are reported to respond with higher rates than those with receptor negative breast cancer (13, 14). However, since some patients with ER+/PR+ breast cancer will not respond to Tamoxifen™ administration, stratification of Tamoxifen™-responsive from unresponsive patients would improve clinical management of breast cancer.

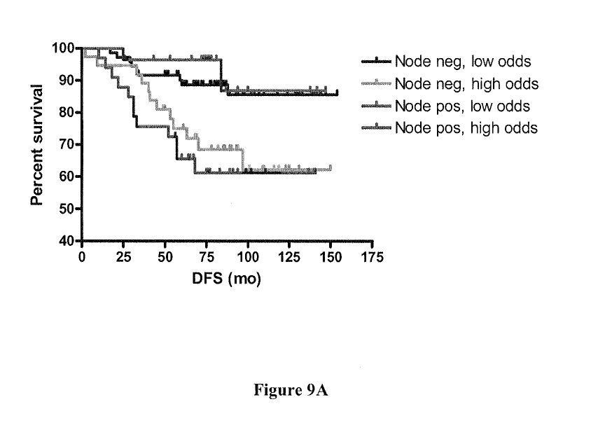

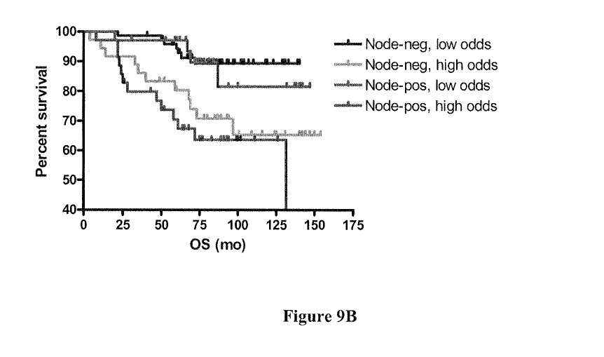

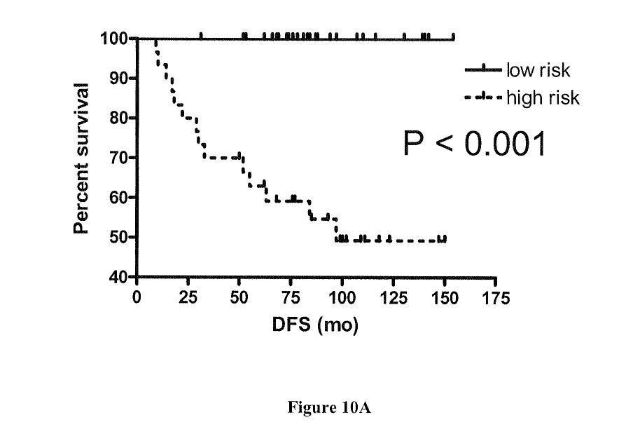

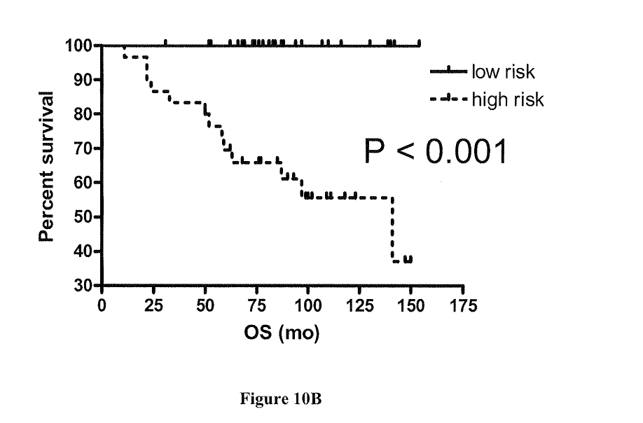

Although no single gene independently correlated with disease-free survival, a ten-gene model was obtained using multivariate Cox regression (Table 12). As shown in FIGS. 9A and 9B, there was a highly significant separation between the low risk (n=30) and high risk (n=30) groups, with 100% of the low risk patients remaining disease-free after 10 years (FIG. 9A). Using the same risk groups defined by this model, analysis of overall survival illustrated similar results (FIG. 9B). These data are highly significant (P<0.001), indicating this gene subset represents a powerful molecular test of ER+/PR+ biopsies for predicting a breast cancer patient's response to Tamoxifen™. Separating the Tamoxifen™-responsive from unresponsive patients with ER+/PR+ biopsies significantly impacts clinical management.

TABLE 12
Multivariate Cox Regression Analyses in ER+, Tamoxifen ™-treated
Population
DFS model (n = 60)

| Gene | Exp (B) = HR | P value |
|---|---|---|
| ER | 1.58 | 0.08 |
| PR | 0.51 | 0.004 |
| BCL2a | 5.54 | 0.01 |
| ERBB4 total | 1.86 | 0.53 |
| ERBB4 JM-a | 0.37 | 0.34 |
| RERG | 0.22 | 0.02 |
| CD34 | 0.13 | 0.04 |
| EDG1 | 0.42 | 0.20 |
| NQO1 | 0.60 | 0.10 |
| PTGDS | 1.56 | 0.11 |

The methods of the invention detect gene expression of Hs.150749 (BCL-2 α), Hs.21099 (CAXII), Hs.390729 (ERBB4), Hs.79136 (LIV-1), Hs.199487 (RERG), Hs.374990 (CD34), Hs.154210 (EDG-1), Hs.406515 (NQO-1), Hs.446429 (PTGDS), Hs.522891 (SDF-1), Hs.208124 (ESR1), Hs.32405 (PGR), Hs.488293 (EGFR) and Hs.446352 (HER-2). Detection of expression of the genes can be useful to assess the prognosis (e.g., likelihood of recurrence) of early stage, ER-positive breast cancers. This was especially valuable in identifying high risk subpopulations of breast cancer patients that are positive for both ER and PR. The methods described herein provide a molecular basis for breast cancer therapeutics by providing individualized assessment of prognosis and/or treatment response. As a result, breast cancer patients may be given therapy regimens tailored to their individual disease (personalized medicine) that more accurately impacts the molecular status (e.g., proliferative activity) of the lesion, rather than being given generic therapies that must be continuously adjusted according to unpredictable responses.

### REFERENCES

1. Wittliff J L, Ma X J, Wang W et al. Expression of Estrogen Receptor-associated Genes in Breast Cancer Cells Procured by Laser Capture Microdissection Correlate with Clinical Outcome. Jensen Symposium, 81. (2003).
2. Ma X J, Wang W, Salunga R et al. Gene Expression Associated with Clinical Outcome in Breast Cancer via Laser Capture Microdissection. Breast Cancer Res Treat 2003; 82(S15).
3. Jin, V X, Sun, H, Pohar, T T, Liyanarachchi, S, Palaniswamy, S K, Hunag, T H, Davuluri, R V. ERTargetDB: an integral information resource of transcription regulation of estrogen receptor target genes, J Mol Endocrinol 2005, 35(2), 225-30.
4. Britton D J, Hutcheson I R, Knowlden J M et al. Bidirectional cross talk between ERalpha and EGFR signalling pathways regulates tamoxifen™-resistant growth. Breast Cancer Res Treat 2006, 96(2):131-46.
5. Glaros S, Atanaskova N, Zhao C, Skafar D F, Reddy K B. Activation function-1 domain of estrogen receptor regulates the agonistic and antagonistic actions of tamoxifen™. Mol Endocrinol 2006, 20(5):996-1008.
6. Levin E R. Bidirectional signaling between the estrogen receptor and the epidermal growth factor receptor. Mol Endocrinol 2003, 17(3):309-17.
7. Junttila T T, Sundvall M, Maatta J A, Elenius K. Erbb4 and its isoforms: selective regulation of growth factor responses by naturally occurring receptor variants. Trends Cardiovasc Med 2000, 10(7):304-10.
8. Srinivasan R, Gillett C E, Barnes D M, Gullick W J. Nuclear expression of the c-erbB-4/HER-4 growth factor receptor in invasive breast cancers. Cancer Res 2000, 60(6):1483-7.
9. Zhu Y, Sullivan L L, Nair S S et al. Coregulation of Estrogen Receptor by ERBB4/HER4 Establishes a Growth-Promoting Autocrine Signal in Breast Tumor Cells. Cancer Res 2006, 66(16):7991-8.
10. Naresh A, Long W, Vidal G A et al. The ERBB4/HER4 intracellular domain 4ICD is a BH3-only protein promoting apoptosis of breast cancer cells. Cancer Res 2006, 66(12):6412-20.
11. Dupont W D. Statisical modeling for biomedical researchers: a simple introduction to the analysis of complex data. Cambridge, UK: Cambridge University Press; 2002.
12. Wittliff J L, Pasic R, Bland K I. Steroid and peptide hormone receptors: methods, quality control, and clinical use. In: Bland K I, Copeland E M, editors. The Breast: Comprehensive Management of Benign and Malignant Diseases. Philadelphia: W.B. Saunders Co.; 1998. p. 458-498.
13. Fisher, B., Redmond, C., Brown, A., Womark, N., Wittliff, J. L. et al. Treatment of Primary Breast Cancer with Chemotherapy and Tamoxifen™. New England J. Med., 305:1-6, 1981.
14. Fisher, B., Redmond, C., Brown, A., Wickerham, D. L., Wolmark, N., Allegra, J. C., Escher, G., Lippman, M., Savlov, E., Wittliff, J. L. and Fisher, E. R. et al. Influence of Tumor Estrogen and Progesterone Receptor Levels on the Response to Tamoxifen™ and Chemotherapy in Primary Breast Cancer. J. Clin. Oncol., 1:227-241, 1983.

While this invention has been particularly shown and described with references to example embodiments thereof, it will be understood by those skilled in the art that various changes in form and details may be made therein without departing from the scope of the invention encompassed by the appended claims.

What is claimed is:

1. A method of optimizing treatment of a human having an estrogen-receptor positive breast cancer, comprising the step of determining a level of expression of genes selected from the group consisting of ESR1, BCL-2 α, ERBB4, ERBB4 JM-a, RERG, CD34, EDG-1, NQO-1, PGR and PTGDS in a breast cancer tissue sample from the human, wherein underexpression of PGR, ERBB4 JM-a, RERG, CD34, EDG-1 and NQO-1 in the sample in combination with overexpression of ESR1, BCL-2 α, ERBB4 and PTGDS in the sample thereby identifies a human that would potentially benefit from a therapy that is alternative to or in combination with a selective estrogen receptor modulator.

2. The method of claim 1, wherein the estrogen-receptor positive cancer is also positive for a progesterone-receptor.

3. The method of claim 1, further including the step of administering at least one alternative therapy alone or in combination to the human with the selective estrogen receptor modulator, thereby treating the human for the estrogen-receptor positive breast cancer.

4. The method of claim 3, wherein the alternative therapy is administered to the human.

5. The method of claim 1, wherein the alternative therapy includes administration of at least one selective estrogen receptor modulator.

6. The method of claim 5, wherein the selective estrogen receptor modulator includes at least one member selected from the group consisting of 2-[4-[1,2-di(phenyl)but-1-enyl]phenoxy]-N,N-dimethylethanamine; [6-hydrox-2-(4-hydroxyphenyl)-1-benzothiophen-3-yl]-4-(2-piperidin-1-ium-1-ylethoxy)phenyl]methanone chloride and (Z)-4-chloro-1,2-diphenyl-1-[4-(2-(N,N)-dimethylamine)ethoxy]phenyl-1-butylene to the human.

7. The method of claim 3, wherein the alternative therapy includes administration of at least one aromatase inhibitor to the human.

8. The method of claim 3, wherein the alternative therapy is administered to the human in combination with the estrogen receptor antagonist.

9. The method of claim 1, wherein the estrogen-receptor positive breast cancer is a primary estrogen-receptor positive breast cancer.

10. The method of claim 1, wherein the estrogen-receptor positive breast cancer is a secondary estrogen-receptor positive breast cancer.

11. The method of claim 1, wherein the breast cancer tissue sample is a laser capture microdissection breast tissue sample.

12. The method of claim 1, wherein the breast cancer tissue sample is an intact tissue section breast tissue sample.

13. The method of claim 1, wherein expression of the genes are identified by a nucleic acid amplification method.

14. The method of claim 1, wherein the breast cancer tissue sample is obtained from a pre-menopausal human.

15. The method of claim 1, wherein the breast cancer tissue sample is obtained from a post-menopausal human.

16. The method of claim 1, wherein expression is identified by measuring messenger RNA levels of the genes.

17. The method of claim 1, wherein the human is lymph node negative for the estrogen-receptor positive breast cancer.

18. The method of claim 1, wherein the human is lymph node positive for the estrogen-receptor positive breast cancer.

19. A method of optimizing treatment of a human having an estrogen-receptor positive breast cancer, comprising the step of determining a level of expression of genes selected from the group consisting of BCL-2 α, CAXII, ERBB4 and RERG in a breast cancer tissue sample from the human, wherein underexpression of the genes in the sample thereby identifies a human that has an increased likelihood of recurrence of the estrogen-receptor breast cancer that would potentially benefit from a therapy to decrease the likelihood of recurrence of the estrogen-receptor positive breast cancer.

20. The method of claim 19, further including the step of determining expression of PGR in the sample, wherein underexpression of PGR in the sample in combination with underexpression of BCL-2 α, CA XII, ERBB4 and RERG identifies a human that would benefit from the therapy.

21. The method of claim 19, wherein the ERBB4 gene is an JM-a ERBB4 variant.

22. The method of claim 19, wherein treatment of the human increases the likelihood of survival of the human.

23. The method of claim 19, wherein the human is lymph node positive for the estrogen-receptor positive breast cancer.

24. The method of claim 19, wherein the human is lymph node negative for the estrogen-receptor positive breast cancer.

25. A method of optimizing treatment of a human having an estrogen-receptor positive and progesterone-positive breast cancer, comprising the step of determining a level of expression of genes selected from the group consisting of PGR, CAXII, ERBB4, ERBB4 JM-a and RERG in a breast cancer tissue sample of the human, wherein underexpression of the genes in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and progesterone-receptor positive breast cancer.

26. A method of optimizing treatment of a human having an estrogen-receptor positive and progesterone-receptor positive breast cancer, comprising the step of determining a level of expression of genes selected from the group consisting of HER2, CAXII, ERBB4 JM-a and LIV 1 in a breast cancer tissue sample of the human, wherein
a) the breast tissue sample is at least one member selected from the group consisting of a stage 1 breast cancer tissue sample and a stage 2 breast cancer tissue sample; and
b) underexpression of CAXII and ERBB4 JM-a in the sample in combination with overexpression of HER2 and LIV 1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and progesterone-receptor positive breast cancer.

27. A method of optimizing treatment of a human having an estrogen-receptor positive and progesterone-receptor positive breast cancer, comprising the step of determining a level of expression of genes selected from the group consisting of ESR1, CAXII, ERBB4, CD34 and EDG1 in a breast cancer tissue sample of the human that is node-negative for the estrogen-receptor positive and progesterone-receptor positive breast cancer, wherein overexpression of ESR1 and CD34 in the sample in combination with underexpression of CAXII, ERBB4 and EDG1 in the sample thereby identifies a human having an increased likelihood of recurrence of the estrogen-receptor positive and progesterone-receptor positive breast cancer that would potentially benefit from a therapy to decrease the likelihood of recurrence of the estrogen-receptor positive and progesterone-receptor positive breast cancer.

28. A method of optimizing treatment of a human having an estrogen-receptor positive and progesterone-receptor positive breast cancer, comprising the step of determining a level of expression of genes selected from the group consisting of HER2, BCL2a, CAXII, CD34, EDG1 and NQO1 in a breast cancer tissue sample of the human that is lymph node-positive for the estrogen-receptor positive and progesterone-receptor positive breast cancer, wherein overexpression of HER2, BCL2a and EDG1 in the sample in combination with underexpression of CD34, CAXII and NQO1 in the sample thereby identifies a human that has an increased likelihood of recurrence of the estrogen-receptor positive and progesterone-receptor positive breast cancer that would potentially benefit from a therapy to decrease the likelihood of recurrence of the estrogen-receptor positive and progesterone-receptor positive breast cancer.


SEQUENCE LISTING (30 sequences)

SEQ ID NO: 1 - DNA, 24 bp, Artificial Sequence
  Primer
gccaaattgtgtttgatggattaa

SEQ ID NO: 2 - DNA, 27 bp, Artificial Sequence
  Primer
gacaaaaccgagtcacatcagtaatag

SEQ ID NO: 3 - DNA, 21 bp, Artificial Sequence
  Primer
agagcactggatgctgttgct

SEQ ID NO: 4 - DNA, 19 bp, Artificial Sequence
  Primer
tggcttagggcttggcttt

SEQ ID NO: 5 - DNA, 21 bp, Artificial Sequence
  Primer
gcacattttgggaagttgcat

SEQ ID NO: 6 - DNA, 26 bp, Artificial Sequence
  Primer
atttctgctcaaagggacaatattct

SEQ ID NO: 7 - DNA, 22 bp, Artificial Sequence
  Primer
aaaagcgacccattcagagact

SEQ ID NO: 8 - DNA, 30 bp, Artificial Sequence
  Primer
aaaaactaaacagaaaagcactctgtacaa

SEQ ID NO: 9 - DNA, 22 bp, Artificial Sequence
  Primer
gccccaaaaggagaagaacatc

SEQ ID NO: 10 - DNA, 19 bp, Artificial Sequence
  Primer
ttctgcccctgccaaatct

SEQ ID NO: 11 - DNA, 19 bp, Artificial Sequence
  Primer
caggcgcaactcctccatt

SEQ ID NO: 12 - DNA, 22 bp, Artificial Sequence
  Primer
ggtcggttccttctcagtcatg

SEQ ID NO: 13 - DNA, 20 bp, Artificial Sequence
  Primer
gtcctgtactggccgttgct

SEQ ID NO: 14 - DNA, 21 bp, Artificial Sequence
  Primer
agcatctgccgtcacattgtt

SEQ ID NO: 15 - DNA, 21 bp, Artificial Sequence
  Primer
ggccattccactttaccacaa

SEQ ID NO: 16 - DNA, 22 bp, Artificial Sequence
  Primer
cagaatgaagagcccaccaatt

SEQ ID NO: 17 - DNA, 22 bp, Artificial Sequence
  Primer
gcaggctgtcctttataatgca

SEQ ID NO: 18 - DNA, 22 bp, Artificial Sequence
  Primer
tgaaaattcctgttgccattcc

SEQ ID NO: 19 - DNA, 21 bp, Artificial Sequence
  Primer
ctccaggcaggttagcacaga

SEQ ID NO: 20 - DNA, 22 bp, Artificial Sequence
  Primer
aggcagagcactcgtaaaaagc

SEQ ID NO: 21 - DNA, 20 bp, Artificial Sequence
  Primer
ctccagaaacggccattcag

SEQ ID NO: 22 - DNA, 20 bp, Artificial Sequence
  Primer
cccacctagccgagtcacaa

SEQ ID NO: 23 - DNA, 21 bp, Artificial Sequence
  Primer
ctcttctgcaccacggtcttc

SEQ ID NO: 24 - DNA, 20 bp, Artificial Sequence
  Primer
ctccgagtcctgaccaagga

SEQ ID NO: 25 - DNA, 20 bp, Artificial Sequence
  Primer
aaattcaccgccttctgcaa

SEQ ID NO: 26 - DNA, 21 bp, Artificial Sequence
  Primer
tgttccgtcatgcacttatcg

SEQ ID NO: 27 - DNA, 21 bp, Artificial Sequence
  Primer
gattggaccgagctggaaaac

SEQ ID NO: 28 - DNA, 22 bp, Artificial Sequence
  Primer
cagccgtcagctattgtggata

SEQ ID NO: 29 - DNA, 25 bp, Artificial Sequence
  Primer
gggaaatattccctagaaacttcca

SEQ ID NO: 30 - DNA, 19 bp, Artificial Sequence
  Primer
gagtccagcgaggttgcaa